(19) United States
(12) Patent Application Publication
(10) Pub. No.: US 2008/0023414 A1
(43) Pub. Date: Jan. 31, 2008

(54) Separating Device, In Particular For Bodily Fluids, And Receptacle Equipped With This Separating Device

(76) Inventor: Franz Konrad, Kremsmunster (AT) (AT)

Correspondence Address: WILLIAM COLLARD;COLLARD & ROE, P.C., 1077 NORTHERN BOULEVARD, ROSLYN, NY, 11576, US

(21) Appl. No.: 11/791,800

(22) PCT Filed: Nov. 10, 2005
(86) PCT No.: PCT/AT05/00447

§ 371 Date: Aug. 3, 2007

(30) Foreign Application Priority Data
Nov. 29, 2004 (AT) A 2003/2004

(51) Int. Cl. G01N 33/49; B01L 3/14

(52) U.S. Cl. 210/789

(57) ABSTRACT

The invention relates to a separating device (11) for inserting in an interior (10) of a housing container (5) for constituents to be separated from substances, which separating device (11) comprises at least one base body (41, 74) with end regions (45, 46) spaced apart from one another in the direction of a longitudinal axis (15), between which at least one flow passage (44) extends. Disposed in front of the flow passage (44) in the filling direction in a region facing away from the interior (10) is at least one filter element (43) with terminal ends (105, 106) mutually spaced apart in the direction of the longitudinal axis (15). Several passages (107) are provided in the filter element (43) extending between the terminal ends (105, 106), and the individual passages (107) each have a flow cross-section which, in terms of its size, lies between the constituents to be separated from the substance. The filter element (43) can be pierced by means of a cannula and for the most part closes again when the cannula is removed. The flow passage (44) establishes a flow connection with the passages (107) disposed in the filter element (43). The invention further relates to a housing unit (1) with a separating device (11) as well as a separating method.

The invention relates to a separating device for inserting in an interior of a housing container and a housing unit fitted with it for separating constituents of a substance, such as body fluid, tissue pieces or tissue cultures, of the type outlined in the introductory parts of claims 1, 2, 3 and 109.

Various different treatment methods and devices are known. For this purpose, substances, such as blood for example, are split into a light and a heavy phase. The light phase in the case of blood is the blood serum or blood plasma, for example, whilst the heavier parts of the blood are formed by cellular elements, such as blood cells.

The displaceable plunger in the interior of the blood sample tube is usually designed so that it deforms under the effect of a centrifugal force, especially when the sample is being centrifuged, as a direct result of the centrifugal force, which enables the light constituents to move upwards in the direction of the open end of the tube, which is usually closed by a closure device at this point, whereas the displaceable plunger moves so that it lies on the heavy phase of the blood, in other words the blood cells, and is no longer able to move down in the direction of the closed end of the tube because of them. This enables the blood cells to be exactly separated from the blood plasma and blood serum.

Blood serum is extremely important to medicine because various essential ingredients, such as glucose, cholesterol, calcium, inorganic phosphorous, proteins, uric acid and others can be detected by tests conducted on this blood serum or blood plasma. The analysis data is directly correlated to the health of the tested person from whom the blood sample has been taken.

Such blood sample tubes with a displaceable plunger and methods involving the use of a displaceable plunger are known from patent specifications U.S. Pat. No. 3,508,653 A, U.S. Pat. No. 4,294,707 A and U.S. Pat. No. 6,280,400 B1, for example. For example, U.S. Pat. No. 6,280,400 B1 describes a separating body of an elongate design, with an elastic top part and an elongate bottom part with an orifice extending through it. The elastic top part is held on the internal wall of the housing containers by means of a press-fit seating and during the centrifugation process, one of the two media to be separated is able to pass either between the top part and the internal wall or through a slit provided in the top part. A perfect separation can not be prevented in all applications.

Another separating device for inserting in a housing chamber of a housing unit is disclosed in patent specification U.S. Pat. No. 5,266,199 A, which has an elastic support body, an elastic ring surrounding in it which has a split in its circumferential region and a ball which can be inserted in a flow passage inside the elastic support body in order to provide a seal. The purpose of the elastic ring surrounding the elastic support body is to provide a sealing mechanism between the internal wall of the housing container and the elastic support body when the separating device is in the position in which it is being used. The flow passage disposed in the elastic support body, which extends between the end regions spaced apart from one another in the direction of a longitudinal axis, is closed by the ball which floats on the denser of the constituents in the usage position.

Patent specification EP 0 753 741 A1 discloses a housing unit with a housing container with two ends spaced apart from one another along a longitudinal axis, at least one of which is provided with an opening. The internal dimension of the housing container in the region of the first open end in the plane oriented perpendicular to the longitudinal axis is bigger than the internal dimension in the region of the other end in the same spatial direction in the plane oriented parallel with it. An annular component is also inserted in the open end, which covers the open end face of the housing containers by means of a collar, and a cylindrical wall part projects into at least certain regions of the interior of the housing container. The annular component has a shoulder adjoining the cylindrical wall part and hence a wider cross-section, on which the elastic seal element of the separating device is supported in the initial position. At the center, the separating device has a recess, which is closed by a thin cover plate in the region of the top end of the housing container. The individual components are assembled, in particular the separating device is inserted, in a vacuum chamber because once the separating device has been inserted, it is no longer possible to access the interior without damaging it. In addition, a foil is adhered to the collar-shaped shoulder of the annular component and a cap is fitted. The interior is filled by piercing the thin cover plate of the separating device, the thin foil and optionally the cap as well. As a result of this filling operation, the vacuum in the interior decreases so that air is also sucked into the interior. This is followed by the centrifugation process, during which the separating device moves out of the annular component in the direction of the closed end and moves onwards with its seal element until it is in abutment with the internal surface of the housing container. The speed at which sinking in the mixture and the already separated constituents takes place is determined by the contact force of the elastic seal element on the internal surface. By selecting the density of the separating device as a whole by reference to the constituents of the mixture to be separated, it floats on the dividing surface between the two media of differing density. The lighter medium is able to pass between the internal surface of the housing container and the elastic seal element during the centrifugation process.

Another housing unit with a separating device is disclosed in patent specification EP 1 005 910 A2, which has a housing container of cylindrical design with a virtually constant internal diameter. Disposed at the open end of the housing container is a closure device which can be pierced, on which the separating device virtually lies, including in the initial position. This separating device is made from a flexible, rebounding material, and a sealing device is provided on the external circumference of the separating device to provide a seal with the internal surface of the housing container. A deformable element is also inserted in the interior, which can be pushed against the internal wall of the outer container by the pressure exerted by the medium when subjected to centrifugal force, thereby forming a flow passage between the separating device and the inserted deformed insert part, which assumes a sealing position with the seal elements disposed on the separating device again when the centrifugal force is removed, thereby enabling the separated media to remain separated from one another.

Another separating device and a housing unit with a separating device and a corresponding method are known from patent specification DE 195 13 453 A1, comprising a test-tube type housing container which is closed by a closure device at an open end region and in which a separating device is inserted for keeping the different media of the mixture apart after separation. To prevent the end face of the separating device, which can now only come into contact with one medium, from being contaminated when the interior of the container is being subsequently filled with the mixture, the separating device is provided with an orifice in the middle region, through which the mixture can be introduced into the remaining interior of the housing container. During the subsequent separation process by conventional centrifugation with a radial centrifugal force (rcf) of 1,000 g to 5,000 g—where g stands for force of gravity and 1 g has a value of 9.81 m/s²—one medium separated out from the mixture is transferred through the orifice in the separating device into the region disposed between the seal mechanism and the separating device and sinks in the direction of the closed end of the housing container as a result. In order to prevent the other medium disposed between the closed end and the separating device from moving through the orifice and being able to mix again, a cone-shaped end stop diverging in the direction of the closed end is provided at a height corresponding to the quantity of the other medium which usually remains, against which the separating device on the end stop, which extends through the orifice, runs. As soon as the external diameter of the end stop corresponds to the internal diameter of the orifice, the separating device remains in this position and the orifice is closed by the stop as a result, so that there can be no exchange between or no re-mixing of the two media. The disadvantage of this design variant is that a special tube with an internally lying stop has to be manufactured and there is no guaranteeing a reliable function of the medium separation due to the orifice provided in the separating device. Furthermore, it is not always possible to obtain a permanently sealed closure between the two phases separated from one another.

Other housing units for centrifuging mixtures of at least two different media to be separated, whereby the housing container is provided with a closure device at both terminal end regions are known from patent specification WO 96/05770 A1. Disposed in the interior is a separating device in the form of a sealing disc formed by a gel. During the centrifugation process, this plug of gel migrates due to its specific weight, which is higher than the specific weight of the medium which has the lower specific weight and lower than the specific weight of the medium which has the higher specific weight, due to the centrifugal forces between the two different, mutually separated media acting on it. In this positioned arrangement, therefore, one mixture can be separated from the other medium of the mixture. The disadvantage of this approach is that the shelf life is no longer sufficient for what in many cases is the normal period of usage due to the fact that the separating device is provided in the form of gel.

Other housing units with separating devices for separating mixtures during centrifugation are known from patent specifications U.S. Pat. No. 3,931,018 A, U.S. Pat. No. 3,779,383 A, U.S. Pat. No. 3,849,072 A, U.S. Pat. No. 3,862,042 A, U.S. Pat. No. 3,882,021 A, U.S. Pat. No. 3,887,464 A, U.S. Pat. No. 3,887,465 A, U.S. Pat. No. 3,890,237 A, U.S. Pat. No. 3,891,553A, U.S. Pat. No. 3,894,950 A, U.S. Pat. No. 3,894,951 A, U.S. Pat. No. 3,894,950 A, U.S. Pat. No. 3,897,337 A, U.S. Pat. No. 3,897,340 A, U.S. Pat. No. 3,897,343 A, U.S. Pat. No. 3,931,010 A, U.S. Pat. No. 3,931,018 A, U.S. Pat. No. 5,632,895 A, U.S. Pat. No. 5,860,937 A, U.S. Pat. No. 6,406,671 B1, U.S. Pat. No. 6,516,953 B1, EP 0 753 741 A1, EP 1 006 360 A2, EP 1 106 250 A2, EP 1 106 251 A2, EP 1 106 253 A2, EP 1 106 252 A2, DE 2 243 569 A.

The underlying objective of the invention is to propose a separating device, a housing unit fitted with such a separating device and a method of separating constituents of a substance, by means of which a separation can be achieved to a high degree of purity and without being susceptible to external influences during the centrifugation process, enabling the mixture to be separated into different media.

This objective is achieved by the invention on the basis of the characterizing features defined in claim 1, because a separating device of this type with a continuous filter of a pre-definable permeability is used, and the flow cross-sections in the filter enable an exact separation into the media to be separated depending on the particle size but this filter nevertheless does not cause an obstruction when taking the blood sample and introducing it into the sample container or housing container of the housing unit, and its function is not detrimentally altered when pierced by a cannula. It is also advantageously possible to dimension the positioning and retention of the separating device in the region of a closure cap of the housing unit so that the separating device remains unchanged in the region immediately around the closure device as it is being pierced by a cannula and when a blood sample is being introduced, and does not move in the direction of the closed end of the blood sample tube until subjected to higher centrifugal forces such as normally occur when centrifuging blood samples, as a result of which the blood plasma and the blood serum is filtered out of the remaining blood cells.

The invention is also independently achieved on the basis of the characterizing features defined in claim 2. The advantages gained as a result of the combination of features defined in this claim reside in the fact that in the operating position, it is exclusively the filter element which assumes the function of bringing about a separation between the two part-chambers on either side of the separating device and there is no need to move the base bodies relative to one another in addition, nor is there any need to close the flow cross-section off. This being the case, the flow passage is formed across only a part-region of the mutually spaced apart end regions, and the filter element is disposed in at least certain regions of the base body. It may therefore be said that the filter element is disposed in the flow passage. This embodiment operates on a so-called two-chamber principle, whereby the filter element cooperating with the base body splits or divides the interior into the two chambers or part-chambers. This embodiment permits the use of a base body of a simple design, which is therefore inexpensive to manufacture but nevertheless offers a high degree of operating reliability in conjunction with the filter element.

The objective of the invention may also be independently achieved on the basis of the characterizing features of claim 3. The advantages achieved as a result of the combination of features defined in this claim reside in the fact that a base body is combined with a separating element disposed on or in it, and the passages provided in the separating element are designed so that all the constituents of the substance to be separated are not able to pass through them until a pressure force or a prevailing pressure differential is applied to the fluid columns to be separated and these forces are acting on them. This being the case, the pressure forces are generated by centrifugal forces but also due to the differences in pressure at each end of the terminal ends. When these pressure forces are removed, the passages prevent any ingress by the constituents to be separated, and once the separation is complete, for example when seeking to obtain serum or plasma, the constituents separated by physical means remain permanently separated. This prevents any subsequent admixing as a result and a high degree of purity is obtained. Furthermore, the separating element which has to be pierced and then re-sealed is no longer needed, which makes the filling process easier and also increases operating reliability.

Also of advantage is another embodiment defined in claim 4 or 5, because only one of the two constituents of the mixture to be separated is able to pass through the filter element in all situations.

Also of advantage is an embodiment defined in claim 6, because it is possible for the lighter medium of the constituents to pass through the filter element for a brief time during which the filter element and the entire separating device is exposed to the centrifugal force, thereby also preventing any such passage for a long storage time.

An embodiment defined in claim 7 or 8 is also of advantage because after piercing and introducing the substance to be separated, the other part-chamber is reliably sealed, thereby pre-venting any back-flow. Furthermore, an expedient choice of the dimension of the height and the associated flow cross-section enables the flow behavior and the filter behavior of the filter element or separating element to be predetermined.

The embodiment defined in claim 9 results in an even more reliable seal of the two part-chambers.

The advantage of the embodiment defined in claim 10 is that the entire interior can be evacuated with the separating device already inserted before closing with the closure device.

Also of advantage is another embodiment as defined in claim 11, because the filter element or the separating element can be more easily adapted to mass tolerances as a result and a more reliable seal is always produced across the entire displacement path in the region of the internal wall of the housing containers.

The advantage of the embodiment defined in claim 12 is that a range of different materials may be used and the separating device as a whole can therefore be adapted to a range of different applications.

As a result of another embodiment defined in claim 13 or 14, in the region of the orifice or pierced orifice made by the cannula, the self-sealing material prevents penetration by one of the two media.

As a result of the embodiment defined in claim 15, the filter element or the separating element can be easily adapted to a range of different usage conditions.

As a result of the embodiment defined in claim 16 or 17, the sealing action between the filter element or separating element and the internal wall can be influenced on the one hand and the closure of the orifice can be achieved by compressing the material at its center on the other hand.

As a result of the embodiment defined in claim 18, a filter element or separating element can be provided which is easy to manufacture.

Also of advantage is an embodiment as defined in claim 19, because it prevents jamming of the base body during the adjusting movement on the one hand and a sealing abutment of the filter element or separating element on the internal wall of the housing container can be achieved on the other hand.

Embodiments as defined in claims 20 to 22 have proved to be of advantage because an even more effective seal can be achieved with respect to the internal wall of the housing container in the region of the filter element or separating element.

As defined in claim 23, a virtually clearance-free abutment of the filter elements or separating element on the sealing stopper is achieved.

As a result of the embodiment defined in claim 24, it is possible on the one hand to provide a filter element or separating element that is easy to manufacture, which can be moved into abutment with the sealing stopper, and on the other hand, during the piercing operation by the cannula, the substance to be introduced into the part-chamber between the separating device and the closure device can be prevented from being sucked in in this region. The bigger this disc-shaped region is, the more reliable the seal is which can be achieved in the peripheral region of the cannula and even deviations occurring during the piercing operation if the cannula is oriented at an angle to the longitudinal axis can be sealed as well.

As a result of the embodiment defined in claim 25 or 26, separating elements of the same dimensions can be easily adapted to different internal dimensions of the housing container. Furthermore, the separating device is easy to manufacture.

Also of advantage is an embodiment defined in claim 27, because it enables a secure, unobstructed abutment of the filter element or separating element on the sealing stopper of the seal mechanism.

The embodiment defined in claim 28 enables the filter element or separating element to be integrated in the base body or bodies.

Also of advantage is another embodiment defined in claim 29, because during the process of moving the filter element or separating element on the internal wall, any constituents of the mixture which might be lodged there can be scraped away, thereby preventing subsequent contamination of the separated serum or plasma in this region as well.

Also of advantage is an embodiment as defined in claim 30, because it enables an even better seal to be achieved with respect to the internal wall in the peripheral region of the filter element or separating element.

As a result of the embodiments defined in claims 31 to 32, a joint movement of the filter element or separating element with the base body is guaranteed.

An even better connection can be achieved between the filter element or separating element and the base body as a result of the embodiment defined in claim 34.

As a result of an embodiment such as defined in claim 35, a standard integral unit comprising several components can be produced, which are easy to manipulate and can be adapted to one another in terms of their function.

Other advantageous options for the connection between the base body and the filter element or separating element are defined in claims 36 to 38, and in this respect it is also possible to combine a whole range of different materials with one another.

The embodiment defined in claim 39 is of advantage because the seal of the separating device or separating element with respect to the internal wall is obtained by means of the housing connecting the base body and the filter element.

As a result of another embodiment defined in claim 40, the constituents of the mixture are reliably prevented from penetrating the peripheral region of the separating device.

Also of advantage is another embodiment defined in claim 41, because an additional separation can be achieved between the part-chambers inside the housing container in the operating position.

Also of advantage is another embodiment defined in claim 42 or 43, because an additional seal can be obtained in the region of the base body with respect to the internal wall of the housing container, thereby enabling a flow to be directed through the flow passage in the base body.

As a result of the embodiment defined in claim 44 or 45, the separating device can be adjusted to accommodate different mixtures to be separated and the sinking rate can also be fixed in addition.

With the embodiments defined in claims 46 to 49, constituents of the mixture to be separated can be reliably prevented from adhering to the separating device and its components. In addition, the flow through the passage can also be improved.

With the embodiments defined in claims 50 and 51, a base body can be provided which is easy to manufacture, and the filter element or separating element can be inserted in it without the need for complex operations so that a unit can be assembled which is simple in terms of its functions and safe to handle.

Also of advantage is another embodiment defined in claim 52 or 53, because the separating device moves reliably, including through the lighter medium. If high density values are selected, the friction which occurs in the region between the internal wall of the container and the separating device can easily be overcome, even with low centrifugal forces, thereby ensuring a reliable movement as far as the operating position.

As a result of other advantageous embodiments defined in claims 54 to 57, the overall density of the separating device can be increased to the degree that rapid and reliable sinking relative to the housing container can be achieved. Furthermore, the density may be varied within a broad range due to the additional insert element, which not only guarantees the sinking movement but also permits a safe release of the hold of the separating device from the retaining mechanism.

Also of advantage is another embodiment defined in claim 58, because a least one flow passage which can be closed is provided between the mutually spaced apart end regions of the separating device, which affords a passage in both directions by means of at least one pressing element in the region of its initial position until the operating position is reached, because the pressing element presses the separating device provided in the form of at least one component against the internal wall of the housing container. Due to this pre-definable pressure, a pre-definable retaining force of the base bodies on the internal wall of the housing container can be fixed. In conjunction with the internal wall of the housing container provided in the form of a control curve, the flow passage constantly narrows during the switch to the operating position, and a full seal is afforded by the base bodies in the operating position.

Also of advantage is an embodiment defined in claim 59, because a reliable flow through the flow passage is always guaranteed for the medium to be separated, even if there are several base bodies, and a reliable seal is also always guaranteed between the housing chambers to be separated inside the housing when the operating position is reached.

Also of advantage is another embodiment defined in claim 60, because the base bodies are only able to effect a movement relative to one another perpendicular to the adjusting movement and any mutual shifting in the direction of the longitudinal axis is prevented.

As a result of the embodiment defined in claim 61, the base bodies can be pressed against respective oppositely lying internal walls of the housing container and thus establish the flow passage between them until the two base bodies also lie forming a tight seal against one another in the mutually facing regions.

The advantage of the embodiment defined in claim 62 or 63 is that the base bodies can be displaced relative to one another inside the housing container in a displacement plane perpendicular to the longitudinal axis during the entire period of usage but always assume the same position relative to one another in the direction of the longitudinal axis. This also makes assembly easier because only a single part has to be inserted in the housing chamber.

Due to another embodiment defined in claim 64, a uniformly directed pressure force can be exerted on the base bodies, thereby preventing any movement out of line or jamming during displacement.

Due to other embodiments defined in claims 65 to 68, the forces which have to be applied in order to effect the movement establishing the flow passage are uniformly transmitted to the base bodies, which on the one hand ensures a reliable contact of the base bodies on the intended points of the internal walls and on the other hand permits unobstructed flow through the flow passage.

As a result of the embodiment defined in claim 69, the separating device can be manufactured easily and inexpensively.

Also of advantage is an embodiment defined in claim 70, because it enables a reliable seal of the flow passage to be obtained, even in the region where the base bodies are in mutual contact with each other.

Also of advantage is an embodiment as defined in claim 71, because an even more reliable seal can be achieved for the housing chambers to be separated between the media to be separated.

As a result of an embodiment as defined in claim 72, dead spaces in the region of the separating device are prevented, which enables a full separation to be obtained without the risk of subsequent contamination of one of the media.

Also of advantage is an embodiment defined in claim 73 or 74, because the separating device only comes into contact with the internal wall of the housing container in the region of the sealing lips and any manufacturing tolerances can therefore be easily compensated.

As a result of an embodiment as described in claim 75, a reliable seal is also obtained between the housing chambers to be separated from one another in the region of the flow passage facing the housing container.

Due to another embodiment defined in claim 76, as perfect seal is obtained under all circumstances in the region provided for the seal.

As a result of another advantageous embodiment defined in claim 77, a large amount of material can be saved.

With an embodiment defined in claims 78 or 79, contact between the base bodies and the internal walls of the housing container over a large surface area is prevented. At the same time, this also affords an adequate guiding action during the entire displacement until a firm seating is obtained. The frictional force needed to produce a firm seating is also increased because a significantly smaller surface is available for contact purposes and manufacturing tolerances are therefore more easily compensated.

Other possible embodiments are defined in claims 80 or 81, whereby the flow passage has a conducive effect on the flow of the mixtures or also on only one of their constituents.

The embodiment defined in claim 82 or 83 permits a mutual orientation of the base bodies in a plane oriented perpendicular to the longitudinal axis, and due to the retaining projection, displacement is restricted when using a pressure mechanism between them and assembly and fitting work in the interior of the housing unit is reduced.

Other advantageous embodiments are defined in claims 84 to 86, which on the one hand enable the base bodies to be joined to one another and on the other hand permit the functions of the pressing element and support element. As a result, it is possible to manage with a small number of base bodies.

Also of advantage is an embodiment defined in claim 87 or 88, because the density values needed by the support body for the displacement can be obtained on the one hand and a reliable closure of the housing chambers to be separated from one another can be achieved in the critical sealing region on the other hand.

Also of advantage is another embodiment such as defined in claim 89. The advantages reside in the fact that a separating device of this type has a base body which can be inserted in an interior of a housing container, and there is always a passage for one of the constituents of the mixture to be separated until the operating position is reached. An automatic mechanical contact or a mechanical seating on the internal surface of the housing container in a pre-definable position is then guaranteed.

As a result of another embodiment defined in claim 90, the gap becomes increasingly smaller during displacement due to the fact that the internal wall is designed as a control curve and a passage through it is left free until the end but a reliable closure of the flow passage can be obtained in the operating position.

Also of advantage are other embodiments defined in claims 91 to 93, because a conical or cone-shaped design of the base body of the separating device produces a sealing contact across virtually its entire contact surface on the internal surface of the housing container.

Due to another embodiment defined in claim 94, a passage for one of the media is reliably prevented between the two mutually spaced end regions of the base body.

Due to the embodiment defined in claim 95, an abutment on the seal element of the closure device is possible, as a result of which the process of filling the interior of the housing unit with the mixture to be separated is significantly improved and facilitated because extensive wetting by the mixture can be prevented in the region of the top portion of the separating device.

Also of advantage is an embodiment defined in claim 96, because the process of filling the interior with the mixture to be separated and also the passage of one of the media through the base body during the centrifugation process are simple.

As a result of an embodiment as defined in claims 97 and 98, a reliable connection can be obtained for a mutual contact in conjunction with an insert part.

An embodiment as defined in claim 99 has proved to be of advantage because it enables the re-setting behavior and hence the contact force on the internal surface of the housing container to be set.

With another advantageous embodiment defined in claim 100, a valve arrangement can be obtained, by means of which a passage for one constituents of the mixture through the separating device can be permitted on the one hand and a sealed separation can also be obtained on the other hand.

As defined in claim 101, the insert part is prevented from moving out of the connection orifice in the direction of the open end of the housing container from the inserted position.

With an embodiment defined in claims 102 or 103, the insert part is prevented from moving out of the connection orifice in the direction of the closed end of the housing container but a flow connection is always maintained between the two end regions of the base body.

Another possible embodiment is defined in claim 104, whereby when the centrifugation process is terminated, a sealed separation can be produced between the two constituents inside the housing container.

The embodiment defined in claim 105 enables the rate at which the separating device sinks through the mixture to be fixed during the centrifugation process.

Also of advantage is an embodiment defined in claim 106, because adapting the position or disposition of the insert part by reference to the base body during the centrifugation process means that it can be fixed, especially if the density of the insert part is slightly higher than the density of the base body, because during the centrifugation process until shortly before it is terminated, a flow is maintained through the connection orifice between the two mutually separated end regions.

Other advantageous embodiments of the insert part are defined in claims 107 and 108, whereby a sealed contact is always guaranteed between the insert part and the connection orifice.

However, the objective of the invention is also achieved by the features specified in claim 109. The advantage of this approach is that in co-operation with the base body or bodies, a reliable seal between the part-chambers to be separated from one another can be achieved inside the interior by means of the filter element in all operating modes of the housing unit. Due to the fact that the flow passage is open in the initial position, a simple option is available for filling the interior of the housing unit with the mixture to be separated, and this can be done after piercing a filter element or separating element disposed in front of the flow passage in the filling direction. The essential aspect is that in the initial position, the base body or bodies of the separating device are retained on the housing container and/or on the closure device with a pre-definable retaining force, thereby ensuring that the entire separating device can not be pushed or moved into the interior. Due to the piercing operation, the filter element is prevented from being contaminated in the part-chamber of the interior which is provided as a means of accommodating the separated serum or plasma under all circumstances, thereby guaranteeing safe storage even for longer periods. When the cannula is pulled out of the filter element or separating element, constituents of the substance to be separated are wiped off the side facing the base body, thereby preventing any undesirable, detrimental effects on the separated serum or plasma. When using the piercable separating device, it is of advantage that a standard blood sample tube can be used, which is closed at one end and can be filled from one end only.

Also of advantage is another embodiment defined in claim 110, because it ensures that the medium to be separated can flow exclusively through the filter element or separating element only in all situations, thereby ensuring operational reliability in all operating modes.

An even more reliable separation of the part-chambers inside the interior is obtained as a result of the embodiment defined in claim 111.

Also of advantage are embodiments as defined in claims 112 to 118, because a predefined retaining force can be guaranteed for the separating device inserted in the interior in the initial position already, before starting the centrifugation process and hence also during the filling process.

Better fixed positioning of the separating device is advantageously obtained in the region of the operating position as a result of the features specified in claims 119 and 120. This being the case, the separating device is able to make contact by means of its end region facing the other end of the housing container or alternatively by means of the seal mechanism on this positioning mechanism, which is preferably provided in the form of a stop surface, on reaching the operating position. This reliably prevents any further movement and hence any associated undesirable mixing in all situations.

Also of advantage is another embodiment defined in claim 121, because the choice of size for the taper or reduction in the internal cross-sectional dimension of the housing container enables the pre-definable displacement path of the separating device to be easily fixed as far as its operating position, in which an all-round, sealed separation is obtained between the interior disposed between the separating device and the closed end or the separating device and the open end of the housing container.

With other advantageous embodiments defined in claims 122 to 124, the housing container can be used for a range of different uses in conjunction with the selected separating devices. If a cylindrical first part-surface is selected, the conditions in terms of dimension will always remain constant across the entire displacement path of the separating device between it and the housing container. This means that a simple and above all reliable displacement can be effected. Furthermore, if a housing container made from plastic is selected, the de-molding ramp can also be dispensed with whilst still guaranteeing constant dimensions across the displacement path.

As a result of the embodiment defined in claim 125, a sealing stopper which is easy to manufacture can be provided on the one hand, which can be mounted in abutment with the filter element or separating element, and during the piercing operation by the cannula, the substance to be introduced into the part-chamber between the separating device and the closure device can be prevented from being sucked in in this region on the other hand. The bigger this disc-shaped region is, the more reliable the seal will be in the peripheral region of the cannula and any deviations which occur during the piercing operation if the cannula is oriented at an angle to the longitudinal axis will also be sealed as well.

Due to the different choice of materials as defined in claims 126 and 127, it is possible to adapt the housing unit to a range of different usage conditions.

Also of advantage is an embodiment defined in claim 128, because the degree of negative pressure in the interior of the housing container makes it easier to set the quantity of mixture to be accommodated. The process of reducing the pressure is also facilitated.

Also of advantage is an embodiment defined in claim 129, because it enables the separating device to be inserted in the interior of the housing container irrespective of position and also makes manufacture easy and inexpensive.

Also of advantage are embodiments such as described in claims 130 to 132 because they permit at least one passable flow passage between the mutually spaced end regions of the separating device, and a passage is left free for at least one pressing element in the region of its initial position through to the point at which the operating position is reached in both directions, and the pressing element, which is provided in the form of a separating device comprising at least one base body presses against certain regions of the internal wall of the housing container. Due to the co-operation between the housing chamber which constantly becomes smaller in the direction of the closed end, and the distance between the base bodies or between them and the housing container, a firm mechanical seating or clamping of the separating device is obtained in the region of the internal surface of the housing container due to the dimensions. This fixed seating or clamping can be achieved when the base body or bodies close the flow passage and the internal dimension of the housing chamber in the region of the operating position of the separating device is the same as or smaller than the external circumference of the base body in the same position. This constant reduction of the flow passage is achieved due to the constant taper of the interior of the housing container, which is responsible for closing the flow passage completely in the manner of a control curve. The displacement is caused by the centrifugal force acting on the separating device, and the separation of separating the mixture to be separated into its individual constituents on the one hand and the displacement on the other hand continue until the pre-definable mechanical stoppage between the separating device and the housing container is reached. In this respect, the length of the displacement path in the direction of the longitudinal axis may be set on the basis of the size chosen for the flow passage in the region of the initial position and the degree of tapering of the interior. As a result of this embodiment of the separating device, it is already possible to proceed with assembly and insert the separating device in the interior and then run an evacuation and close it by means of the closure device, because access to what in this instance is the closed end is always possible through the separating device. Consequently, filling can proceed unobstructed and there is no need to insert the separating device afterwards or remove the closure device prior to starting the centrifugation process. This ensures a high degree of operating safety.

As a result of the embodiment defined in claim 133, the displacement which must act on the separating device due to the centrifugal acceleration in order to move the separating device from the initial position so that it moves in the direction of the operating position can be fixed.

Due to the embodiment defined in claim 134, the dimensions of the base body of the separating device and the housing container can be exactly adapted.

Finally, the advantage of the embodiment defined in claim 135 is that by enabling the internal dimensions of the housing container to be exactly adapted in conjunction with the gap size, the pre-definable position or disposition of the operating position of the separating device inside the housing unit can be predefined in a simple manner.

However, the objective of the invention is also independently achieved by means of a method of separating constituents to be separated from substances, such as body fluids, tissue pieces or tissue cultures, based on the features specified in claim 136. The advantages gained by the combination of features defined in this claim reside in the fact that there is a possibility whereby the substance to be separated is introduced into the interior of the housing unit and is sucked through the passage of the separating element into the other part-chamber between the separating device and the closed end of the housing container due to the vacuum or negative pressure prevailing in the interior. This avoids piercing or piercing is no longer necessary. The entire housing unit is then centrifuged in a manner known per se, as a result of which the substance is split into the constituents to be separated and the relative shift of the separating device takes place starting from the initial position into the operating position or separating position. The sinking rate during the sinking process may be selected so that it is rapid up to the point of reaching the positioning mechanism because whilst the centrifugal force is effective, all of the constituents to be separated are able to pass through the passages of the separating element. Only when this active force or pressure is removed do the passages prevent ingress by the constituents, thereby ensuring a reliable separation of the part-chambers on either side of the separating device.

Another advantageous approach is described in claim 137, whereby subsequent admixing of the constituents is reliably prevented in both flow directions once the separation is complete.

Also of advantage is another variant of the method defined in claim 138, because further movement of the separating device relative to the housing container is prevented and the pre-definable non-operating position and hence the separation position is clearly fixed.

Finally, another advantageous approach is described in claim 139, whereby depending what chemical is applied, the filled substance and its constituents can be influenced or affected, thereby enabling the subsequent test routines to be run perfectly.

The invention will described in more detail below with reference to examples of embodiments illustrated in the appended drawings.

Of these:

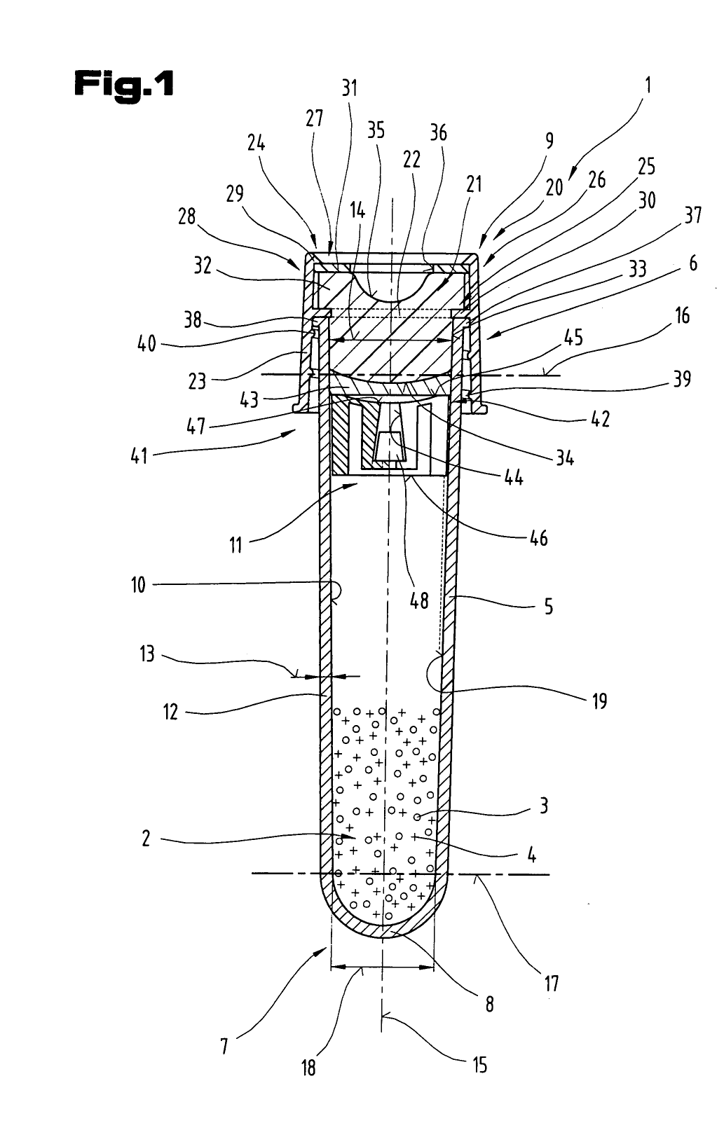

FIG. 1 is a simplified, schematic diagram illustrating a side view in section of a housing unit proposed by the invention with a separating device proposed by the invention in the initial position and a closure device;

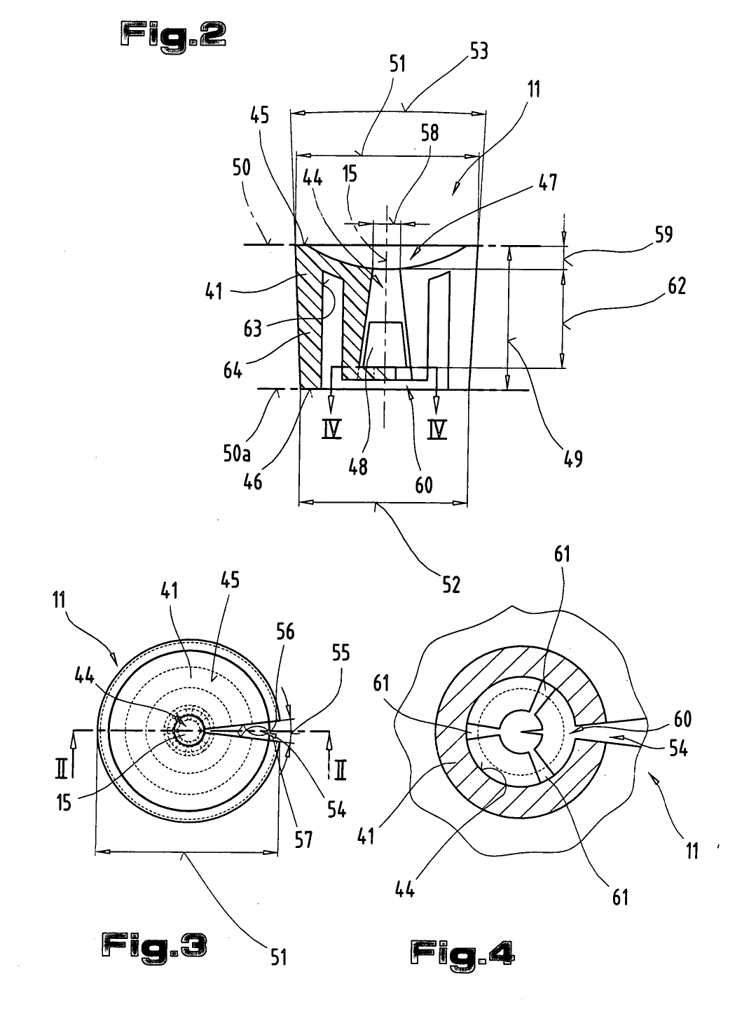

FIG. 2 is a diagram on a larger scale showing a side view of the separating device illustrated in FIG. 1 with the filter element removed, in section along line II-II indicated in FIG. 3;

FIG. 3 is a plan view of the separating device illustrated in FIG. 2;

FIG. 4 is a plan view of a part-region of the base body in the region of the flow passage, viewed in section along line IV-IV indicated in FIG. 2 and on an enlarged scale;

FIG. 5 is a plan view of another embodiment of the retaining mechanism in the base, viewed in section on a larger scale;

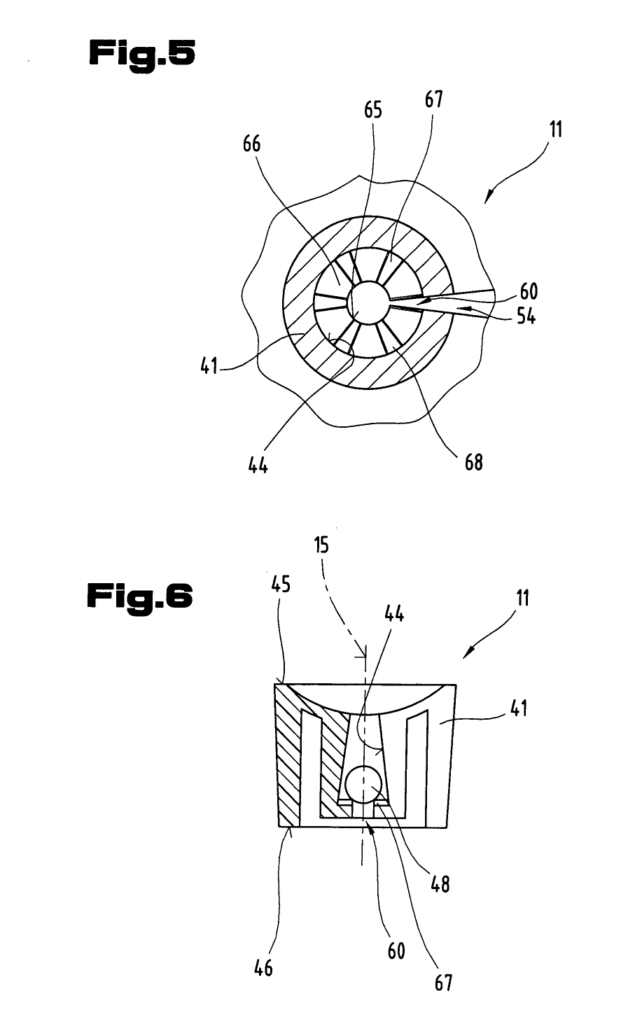

FIG. 6 is a side view in section on a larger scale showing a separating device with the filter element removed but with a different insert part and retaining mechanism;

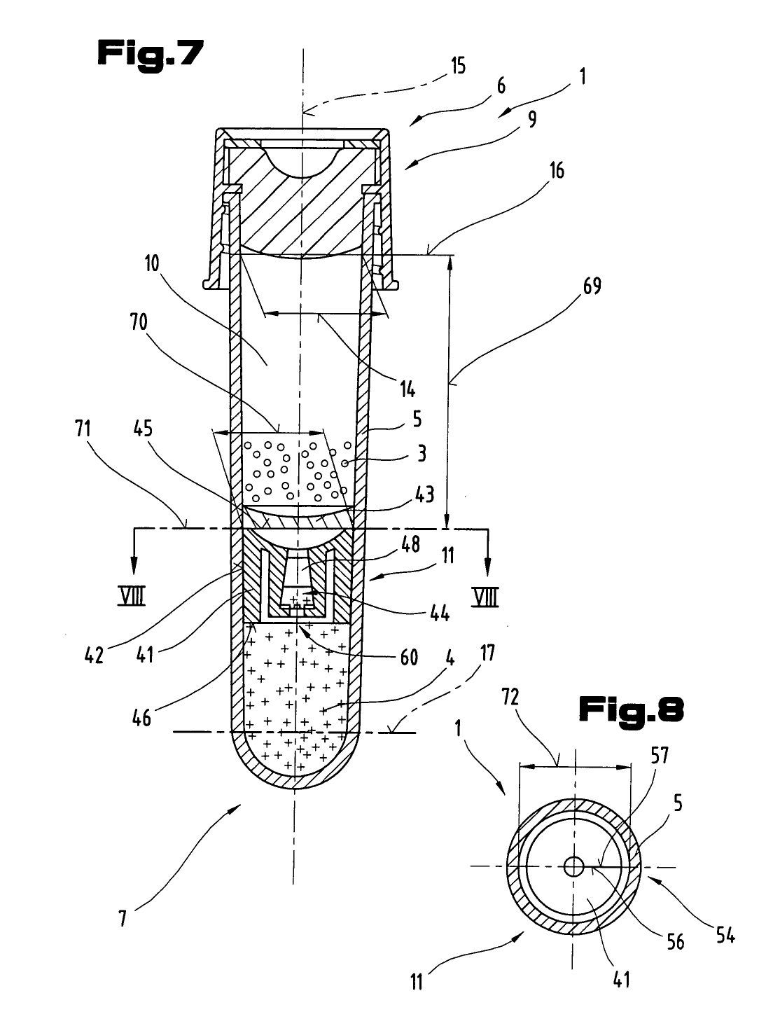

FIG. 7 illustrates the housing unit once separation of the media is complete and with a separating device moved into the operating position;

FIG. 8 is a plan view showing the separating device in the operating position, viewed in section along line VIII-VIII indicated in FIG. 7;

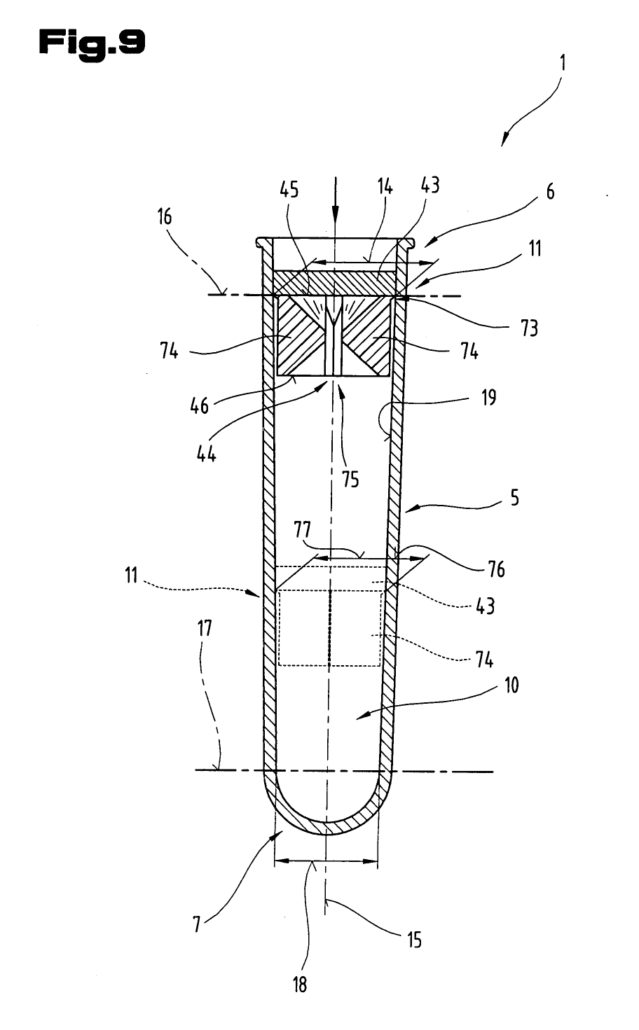

FIG. 9 is a simplified, schematic diagram in section, showing a side view of another housing unit proposed by the invention with another separating device proposed by the invention in the initial position with the closure device removed;

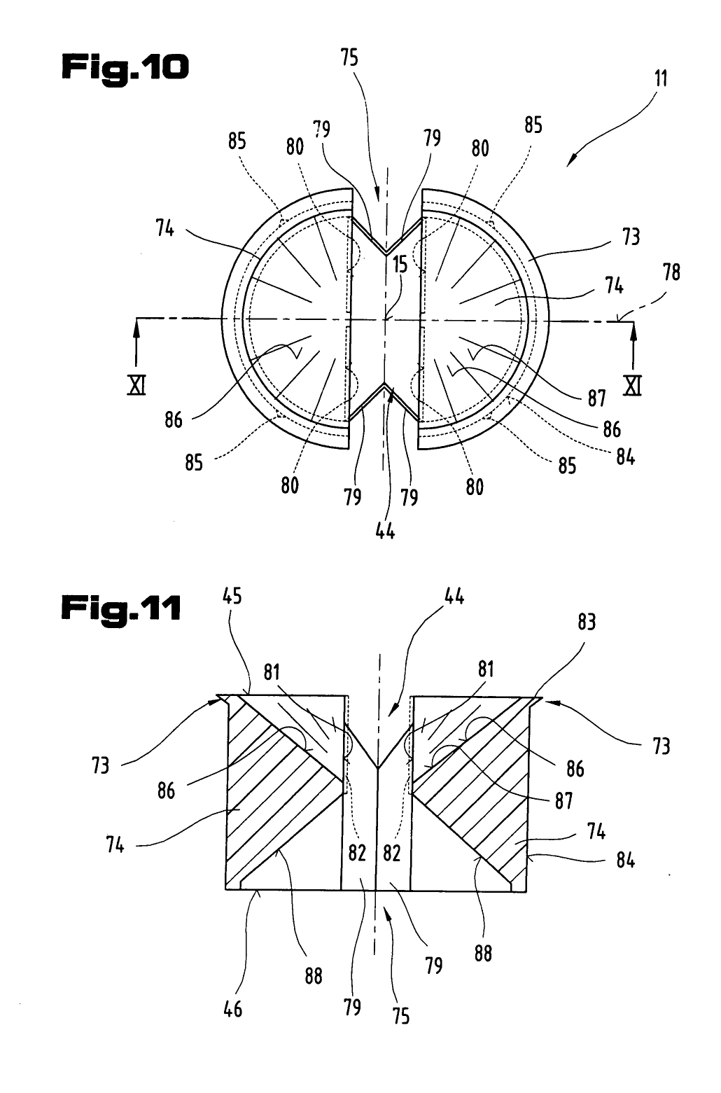

FIG. 10 is a simplified, schematic diagram on a larger scale showing a plan view of the separating device illustrated in FIG. 9 with the filter element removed;

FIG. 11 shows a side view of the separating device illustrated in FIG. 10, viewed in section along line XI-XI indicated in FIG. 11;

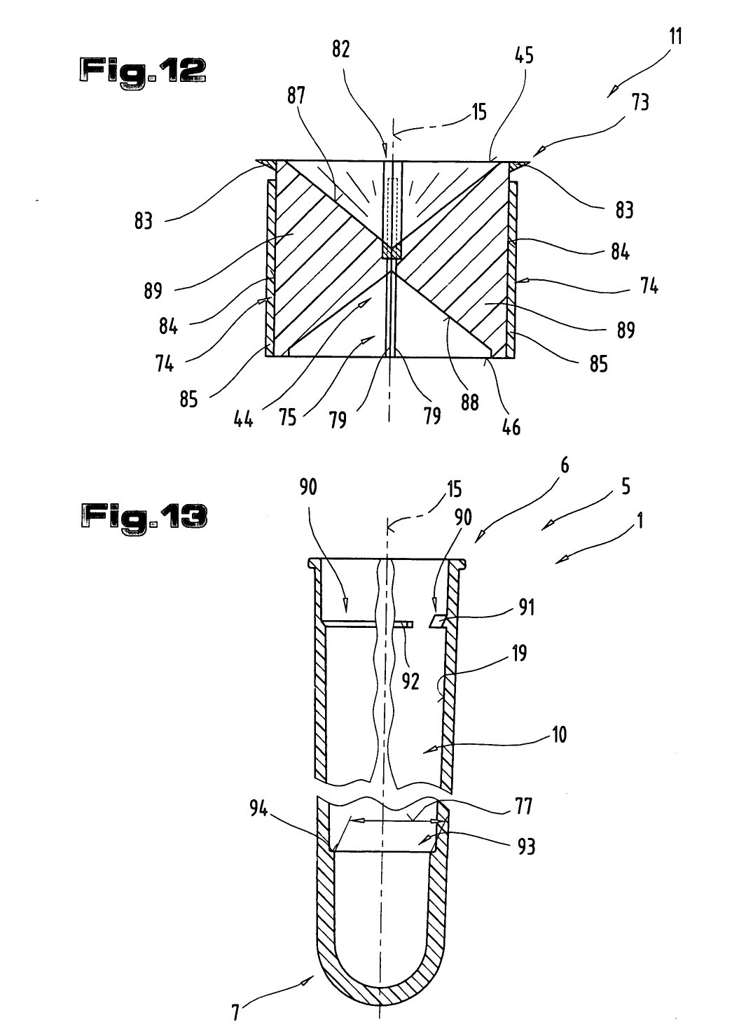

FIG. 12 is a simplified, schematic diagram showing a side view in section of another embodiment of a separating device proposed by the invention with the filter element removed and the flow passage in the closed position;

FIG. 13 is a simplified, schematic diagram showing a side view in section of another embodiment of a housing unit proposed by the invention, with the closure device removed and showing the separating device;

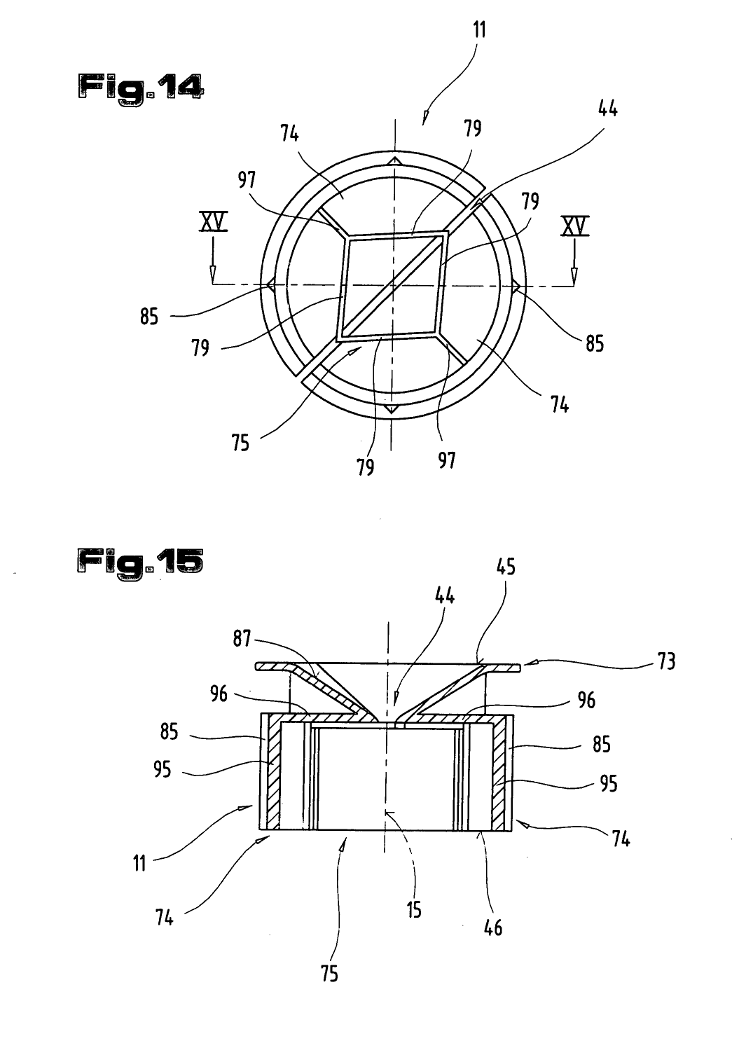

FIG. 14 is a simplified, schematic diagram illustrating another separating device proposed by the invention viewed from underneath;

FIG. 15 shows the separating device illustrated in FIG. 14 with the filter element removed, viewed in section along line XV-XV indicated in FIG. 14;

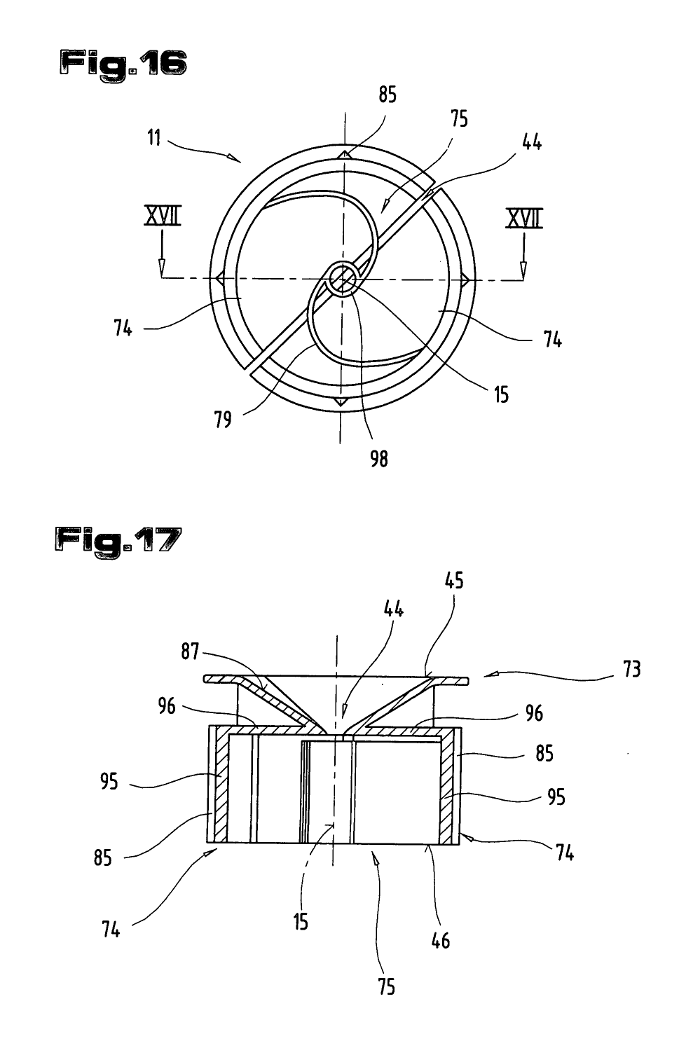

FIG. 16 is a simplified, schematic diagram illustrating another separating device proposed by the invention viewed from underneath;

FIG. 17 shows the separating device illustrated in FIG. 16 with the filter element removed, viewed in section along line XVII-XVII indicated in FIG. 16;

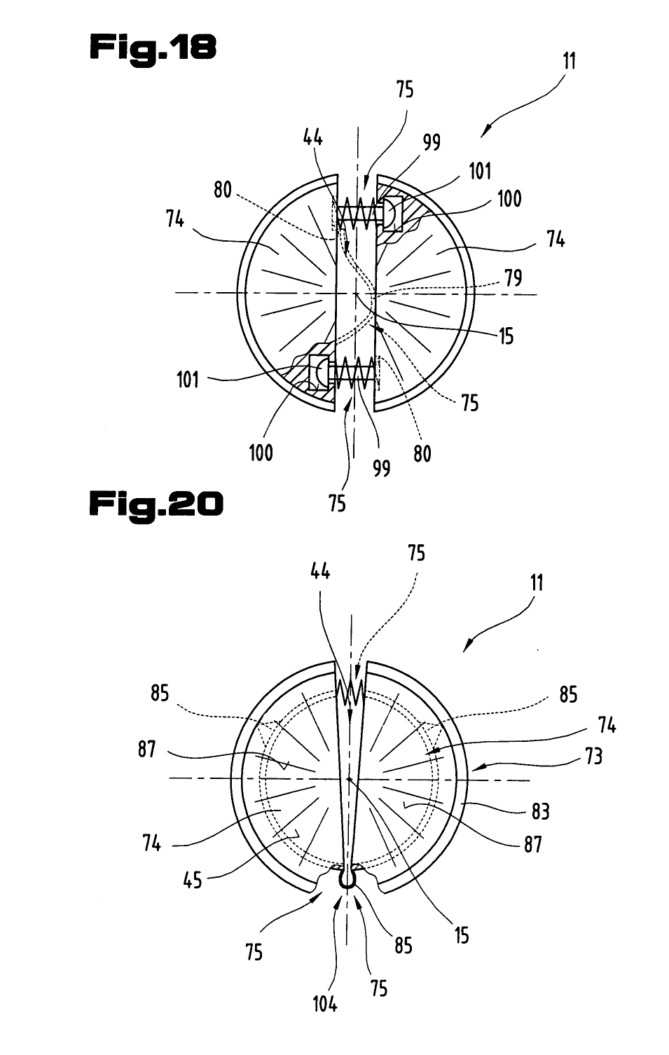

FIG. 18 is a simplified, schematic diagram in partial section showing a plan view of another embodiment of the separating device, with the filter element removed;

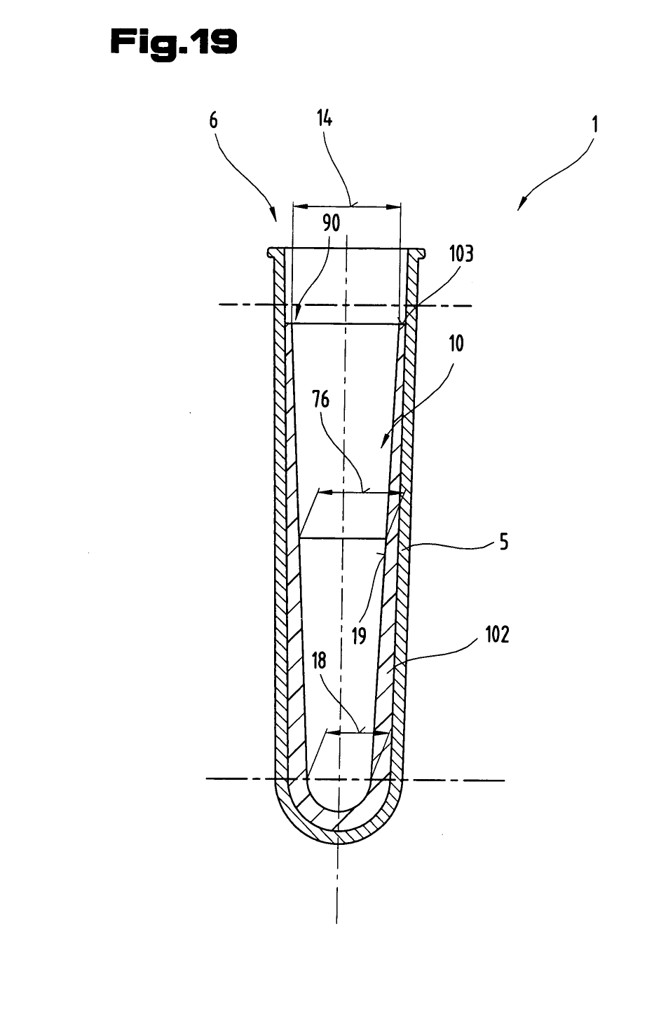

FIG. 19 is a simplified, schematic diagram showing a side view in section of another embodiment of a housing unit proposed by the invention with the closure device removed and showing the separating device;

FIG. 20 is a simplified, schematic diagram showing a plan view in partial section of another embodiment of the separating device proposed by the invention with the filter element removed.

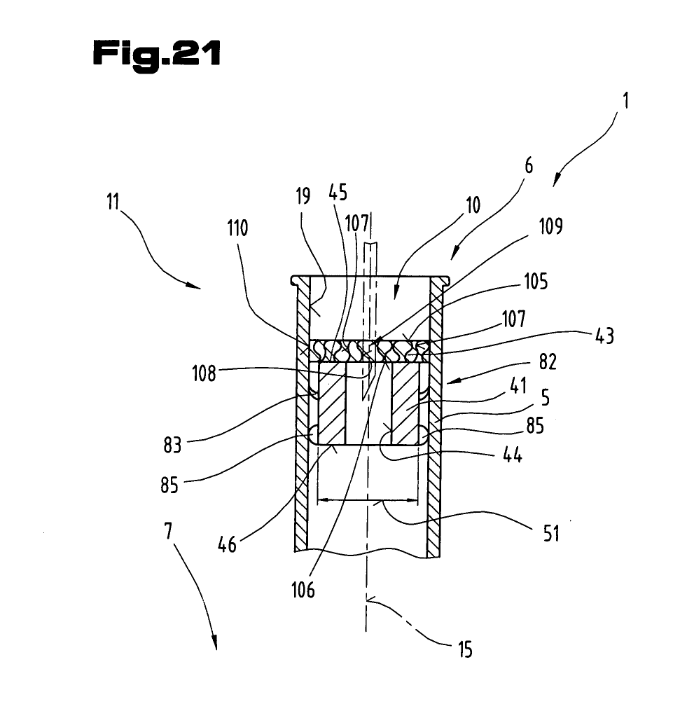

FIG. 21 is a simplified, schematic diagram showing a side view in section of a part-region of another separating device proposed by the invention;

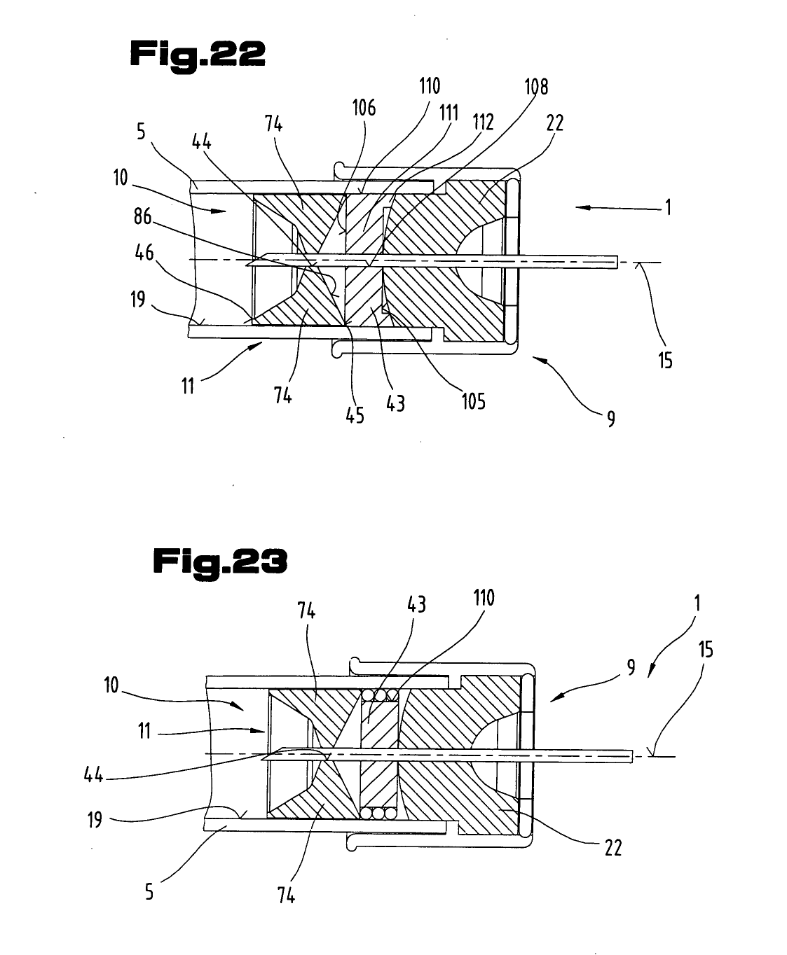

FIG. 22 is a simplified, schematic diagram showing a side view in section of a part-region of a different housing unit;

FIG. 23 is a simplified, schematic diagram showing a side view in section of a part-region of another housing unit;

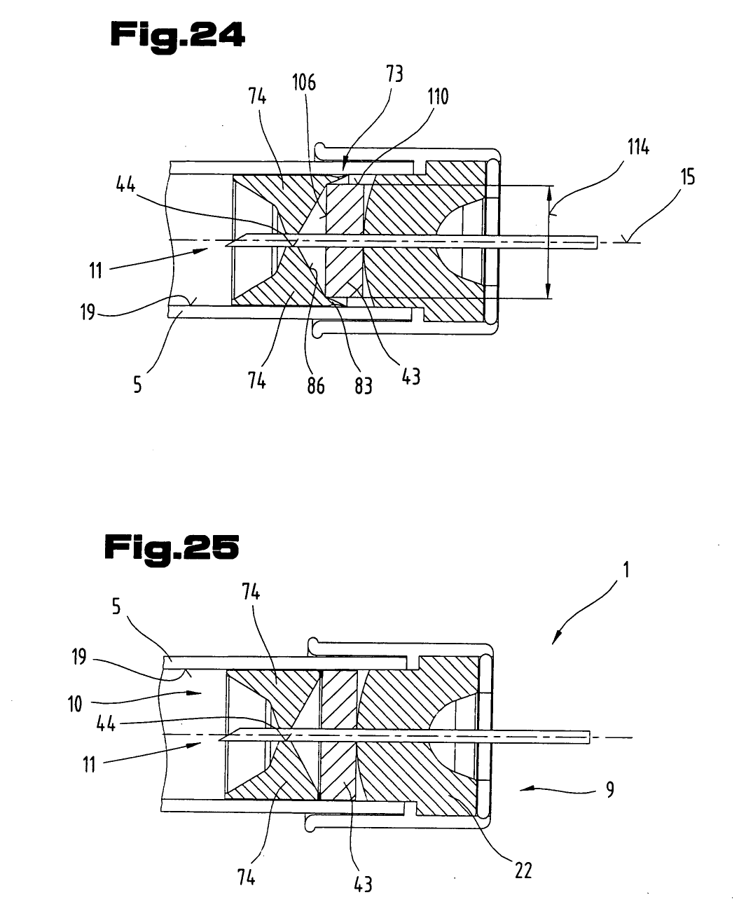

FIG. 24 is a simplified, schematic diagram showing a side view in section of a part-region of another housing unit;

FIG. 25 is a simplified, schematic diagram showing a side view in section of a part-region of another housing unit;

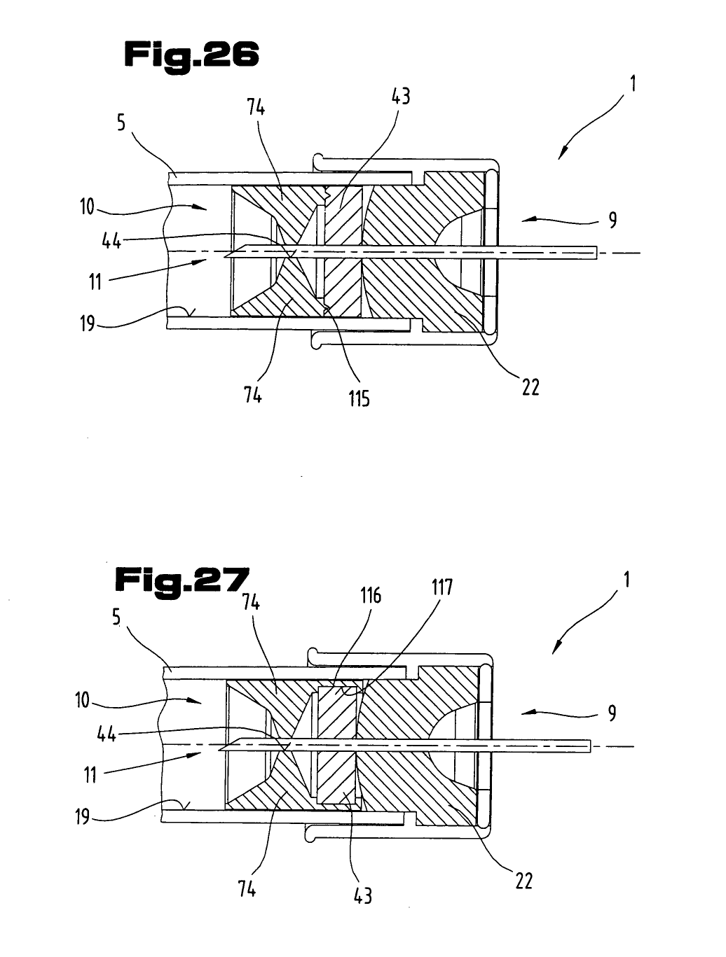

FIG. 26 is a simplified, schematic diagram showing a side view in section of a part-region of another housing unit;

FIG. 27 is a simplified, schematic diagram showing a side view in section of a part-region of another housing unit;

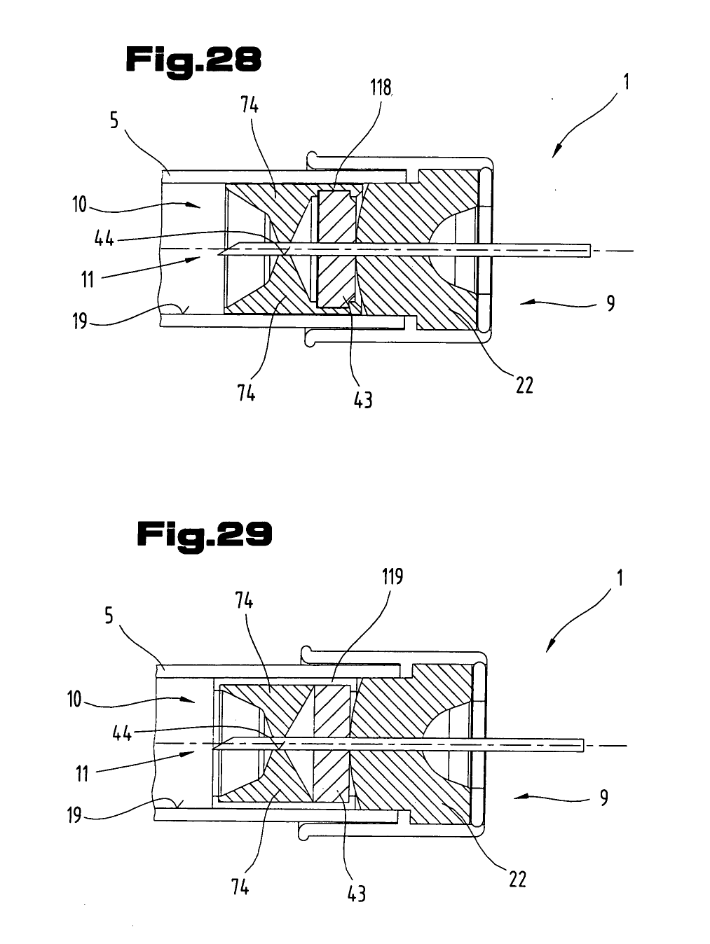

FIG. 28 is a simplified, schematic diagram showing a side view in section of a part-region of another housing unit;

FIG. 29 is a simplified, schematic diagram showing a side view in section of a part-region of another housing unit;

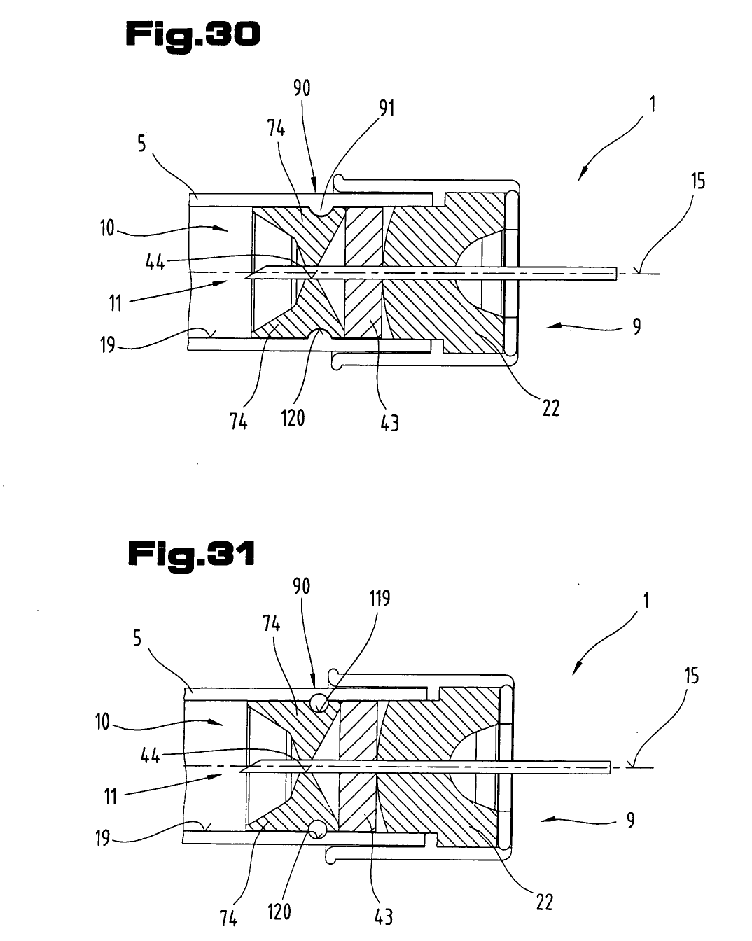

FIG. 30 is a simplified, schematic diagram showing a side view in section of a part-region of another housing unit;

FIG. 31 is a simplified, schematic diagram showing a side view in section of a part-region of another housing unit;

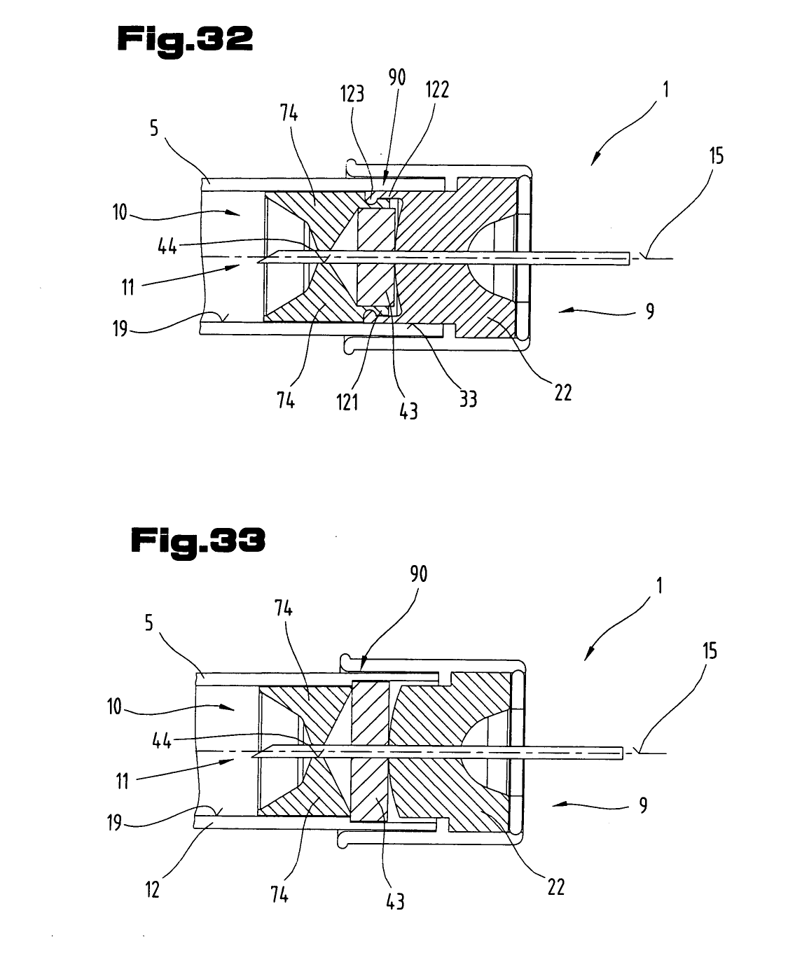

FIG. 32 is a simplified, schematic diagram showing a side view in section of a part-region of another housing unit;

FIG. 33 is a simplified, schematic diagram showing a side view in section of a part-region of another housing unit;

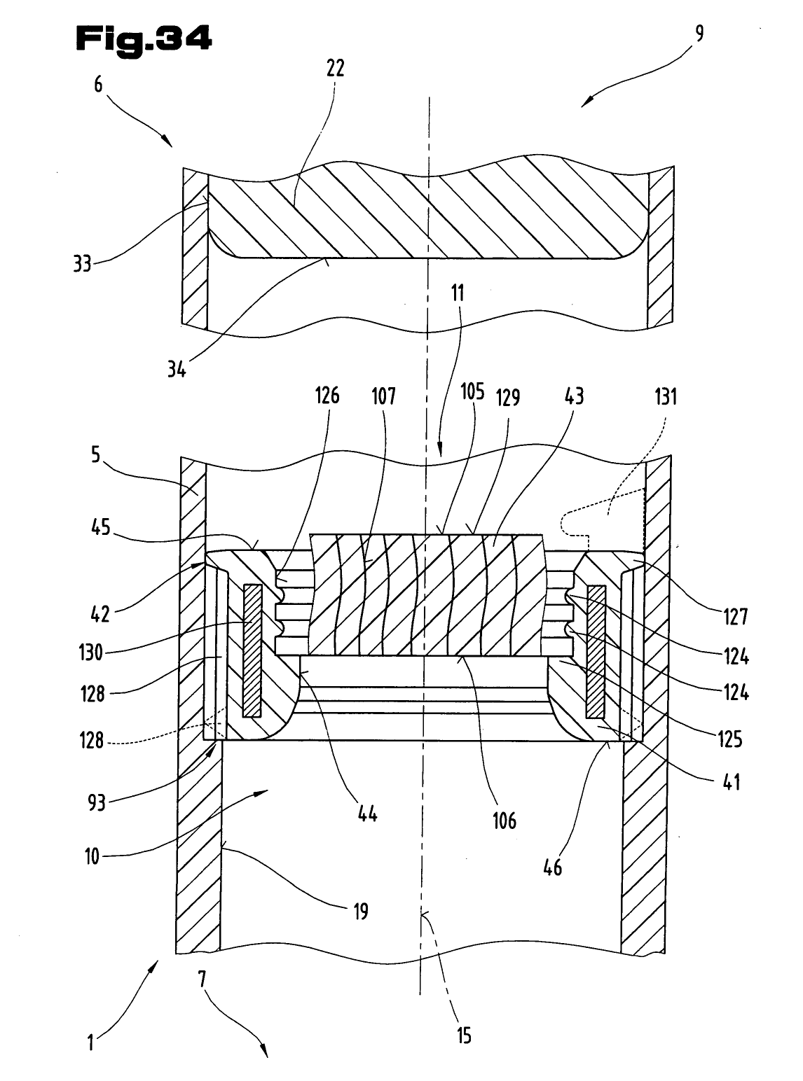

FIG. 34 is a simplified, schematic diagram showing a side view in section of a part-region of another design of housing unit proposed by the invention with a different separating device proposed by the invention disposed in the operating position and a part of a closure device;

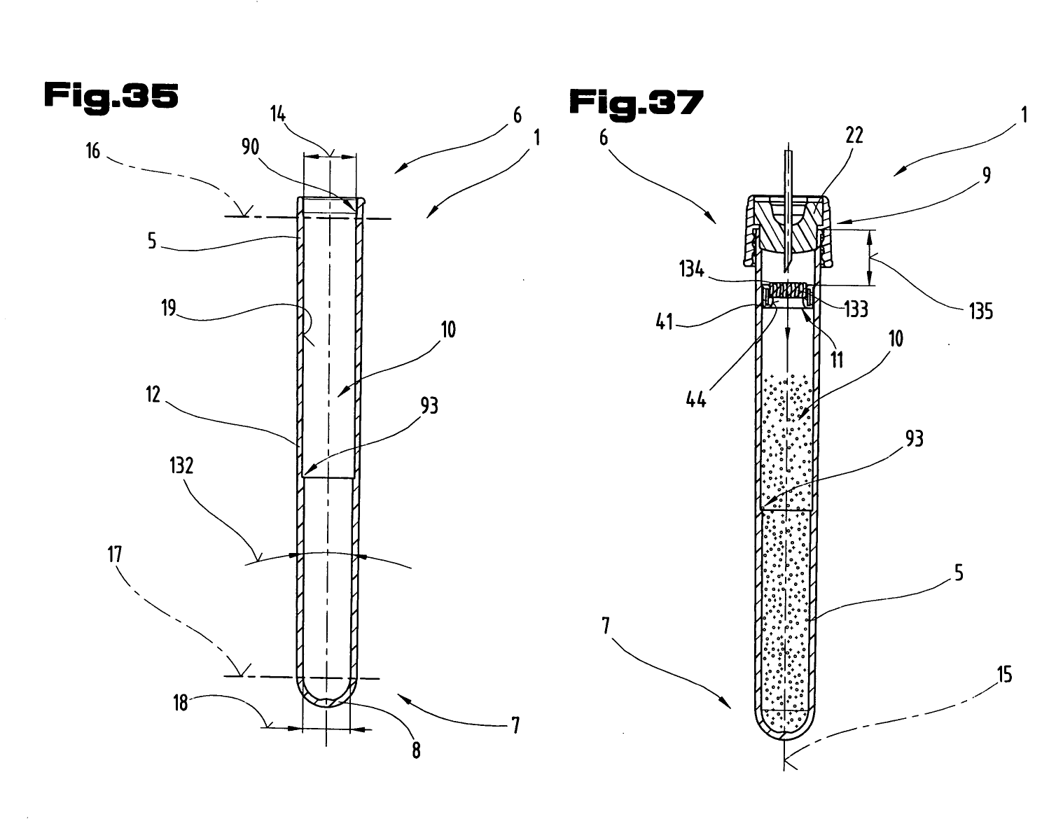

FIG. 35 is a side view in section illustrating one possible embodiment of a housing containers for making up a housing unit;

FIG. 36 is a simplified, schematic diagram showing a side view in section of a part-region of another design of housing proposed by the invention with a different separating device proposed by the invention disposed in the operating position and a part of a closure device;

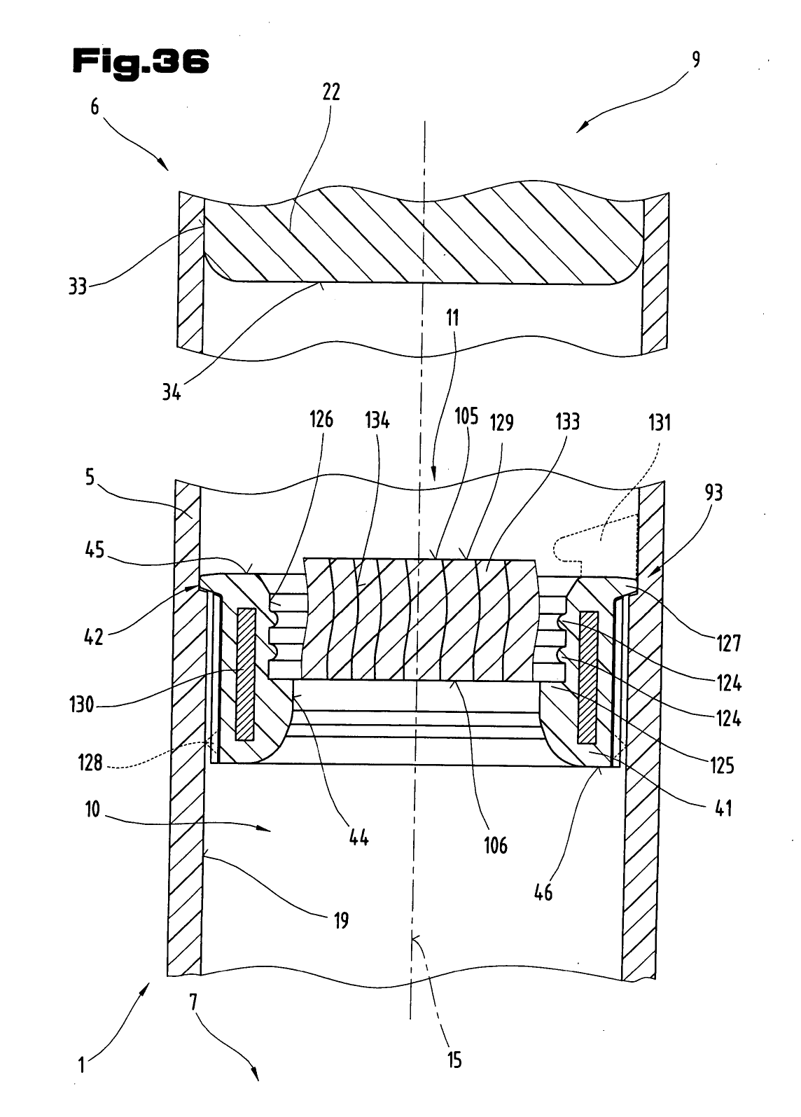

FIG. 37 is a simplified, schematic diagram showing a side view in section of another design of housing unit proposed by the invention with a different separating device proposed by the invention disposed in the initial position.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc,. relate to the drawing specifically being described and can be transposed in terms of meaning to a new position when another position is being described. Individual features or combinations of features from the different embodiments illustrated and described may be construed as independent inventive solutions or solutions proposed by the invention in their own right.

The embodiments illustrated as examples represent possible design variants of the separating device or a housing unit and it should be pointed out at this stage that the invention is not specifically limited to the design variants specifically illustrated, and instead the individual design variants may be used in different combinations with one another and these possible variations lie within the reach of the person skilled in this technical field given the disclosed technical teaching. Accordingly, all conceivable design variants which can be obtained by combining individual details of the design variants described and illustrated are possible and fall within the scope of the invention.

All figures given in respect of ranges of values in this description should be construed as meaning that these and all part-ranges are included, for example when 1 to 10 is specified, this should be construed as meaning that all part-ranges starting form the bottom limit 1 and up to the upper limit 10 are included, i.e. all part-ranges start with a lower limit of 1 or more and end with an upper limit of 10 or less, e.g. 1 to 1.7 or 3.2 to 8.1 or 5.5 to 10.

FIGS. 1 to 4 illustrate a housing unit 1 for a mixture 2 or substances comprising at least two different constituents or media 3, 4, such as body fluids, tissue pieces or tissue cultures, for example, which is designed so that the mixture 2 disposed in the housing unit 1 can be separated into at least two of its constituents. These constituents or media 3, 4 in the case of blood are serum or plasma, for example, as well as cellular constituents (erythrocytes, leukocytes and thrombocytes). The size of the erythrocytes is 7.5×2 μm and they are nucleus-free cells of a disc shape. It is possible to impart a reversible change of shape to these constituents.

Leukocytes include granulocytes, monocytes and lymphocytes. The granulocytes have a segment-shaped or bar-shaped cell nucleus. Neutrophiles have a diameter of between 9 μm and 12 μm, eosinophiles a diameter of between 11 μm and 14 μm and basophiles a diameter of between 14 μm and 16 μm. Monocytes have a kidney-shaped cell nucleus and their size is between 15 μm and 30 μm. Lymphocytes on the other hand, have a rounded nucleus, with a diameter of a size between 7 μm and 9 μm but may also be 12 μm. Thrombocytes are disc-shaped and have a size of 4 μm×0.6 μm and have no nucleus.

This separation or splitting of the mixture 2 into its constituents or media 3, 4 may be performed physically by centrifugation in a conventional manner, for example, and is run starting from the non-operating position until a radial centrifugal acceleration of 1,000 g to 5,000 g, preferably between 1,800 g and 2,200 g, is reached, where g stands for gravitational acceleration and the value of 1 g is 9.81 m/s². As a result, it is possible to separate the more solid phase from the liquid phase or separate them on the basis of the different density values, as will be explained in more detail below with reference to the subsequent drawings illustrating different embodiments.

The housing unit 1 comprises an approximately cylindrical housing container 5 with two mutually spaced ends 6, 7, and in the embodiment illustrated as an example here, the end 6 is open and the end 7 is closed by a terminal wall 8. The end 6, which is open in this instance, can be closed if necessary by a closure device 9 illustrated in a simplified format, which may be of the design specified in patent specifications EP 0 445 707 B1, EP 0 419 490 B1, U.S. Pat. No. 5,275,299 A, U.S. Pat. No. 5,495,958 A and U.S. Pat. No. 5,522,518 A, for example, and for the sake of avoiding repetition, reference may be made to the disclosures relating to the embodiment of the cap, the seal mechanism, the housing or housing container, the coupling mechanism between the cap and the seal mechanism as well as the cap and the housing container 5 and the retaining ring, which disclosures are incorporated in this application by reference. Inserted in an interior 10 surrounded by the housing container 5 is a separating device 11, which is disposed directly adjacent to the closure device 9 in the initial position. The method used for fitting or assembly will be described in more detail below. This housing container 5 with the closure device 9 might also be designed or used as an evacuated blood sampling tube based on a range of different embodiments.

The housing container 5 may be of a bottle-shaped, vial-shaped, plunger-shaped or similar design made from a range of different materials, such as plastic or glass for example. If plastic is selected as the material for the housing container 5, it may be liquid-proof; in particular watertight and optionally impermeable to gas, and may be made from polyethylene terephthalate (PET), polypropylene (PP), polyethylene (PE), polystyrene (PS), high-density polyethylene (PE-HD), acrylonitrile butadiene styrene copolymers (ABS) or similar or a combination of these, for example. The housing container 5 also has a container wall 12 with a wall thickness 13, and the container wall 12 extends from one end 6 with an internal dimension 14 in a plane 16 oriented perpendicular to a longitudinal axis 15 extending between the two ends 6, 7 as far as another plane 17 disposed in the region of the end 7 and parallel with the first plane 16 with a preferably smaller dimension 18. The container wall 12 of the housing container 5 has an internal wall 10 surrounding the interior 10 and an internal surface facing the interior 10 as well as an external surface remote from it, which therefore determines an external circumference for the housing container 5. The internal wall 19 of the container wall 12 with the internal clearance dimension 14, 18 therefore determines an internal cross-section, which may be of different cross-sectional shapes such as circular, elliptical, oval, polygonal, etc., for example. The shape of the external cross-section may also be circular, elliptical, oval, polygonal, etc., although it would also be possible for the shape of the external cross-section to be different form the shape of the internal cross-section.

It is of advantage if the internal dimension 14 of the housing container 5 is designed so that it has a minimal constant reduction compared with the internal dimension 18, starting from one end 6 through to the other end 7 spaced apart from it, so that the housing container 5, if it is made from a plastic material by an injection molding process, can be easily removed from the injection mould. This conical taper between the two planes 16, 17 also determines the degree of reduction in the internal dimension starting from the larger dimension 14 to the smaller dimension 18. The taper or cone angle is between 0.1° and 3.0°, preferably between 0.6° and 0.8°, by reference to the internal oppositely lying surfaces or internal walls 19 of the housing container 5. At this stage, it should be pointed out that the described dimensions for the distance between the oppositely lying internal and external surfaces of the components, the diameter, the circumference along a casing surface or case line as well as the cross-section or the cross-sectional surface relate respectively to planes oriented perpendicular to the longitudinal axis 15 and may always relate to the same spatial direction for determining the dimensions.

As may also be seen from this diagram, the end 6 has an open terminal end, which can be closed by means of the closure device 9 if necessary, and which can be re-opened. To this end, the closure device 9 has a cap 20 surrounding the open terminal end and a seal mechanism 21 retained in it, such as a sealing stopper 22, for example, made from a highly elastic and self-sealing material which can be pierced, such as pharmaceutical rubber, silicone rubber or bromobutyl rubber, for example. This cap 20 is disposed concentrically with the longitudinal axis 15 and is formed by a circular-shaped cap case 23. Disposed between the cap 20 and the seal mechanism 21 are coupling means, such as coupling parts 24 to 27 of a coupling mechanism 28, and in the case of the cap 20 projections 29, 30 are provided extending round at least parts of the internal circumference, and optionally a retaining ring 31, and the seal mechanism 21 has a shoulder 32 projecting out from at least certain regions of its external circumference.

The seal mechanism 21 in the embodiment illustrated as an example here is provided in the form of the sealing stopper 22 and has a circumferentially extending, cylindrical seal surface 33 disposed more or less concentrically with the longitudinal axis 15, which sits in contact with the internal surface of the housing container 5 in its position sealing the portion of the end 6. As a result, the internal surface or the internal wall 19 of the housing container 5 in this region must be of a surface quality good enough to act as a seal surface. The seal mechanism 21 also has another seal surface 34 oriented more or less perpendicular to the longitudinal axis 15, which, in co-operation with the seal surface 33 lying against the internal wall 19, closes and seals the interior 10 of the housing container 5 at its open terminal end from the external atmosphere. Providing the projection 30 between the shoulder 32 extending beyond the seal surface 33 and the open terminal end of the housing container 5 obviates the need to adhere or firmly bond the shoulder 32 directly on the terminal end.

It may also be preferable for the seal mechanism 21 to be provided with a recess 35 on the side facing the retaining ring 31, which has a more or less identical cross-sectional surface to an orifice 36, and this orifice 36 is designed so that, in terms of its dimension, a cannula can be fed through unobstructed, thereby enabling the seal mechanism 21 to be pierced.

The shoulder 32 constituting the coupling part 26, which projects out from the seal surface 33 of the seal mechanism 21 in at least part-regions of the circumference in a flange-type arrangement, is retained between the projections 29 and 30, which are disposed in two planes mutually spaced apart in the direction of the longitudinal axis 15 and perpendicular thereto, and are provided in the form of projections or blocking projections in at least certain regions or alternatively extending circumferentially in a circular shape. To ensure that the seal mechanism 21 is securely retained in the cap 20, it is also possible to insert the retaining ring 31 between the shoulder 32 and the projection 29. This being the case, the retaining ring 31 has a bigger external diameter than an internal dimension between the projections 29 and 30 in the direction perpendicular to the longitudinal axis 15. Likewise, the diameter of the orifice 36 of the retaining ring 31 is smaller than a largest external dimension of the shoulder 32 in a plane perpendicular to the longitudinal axis 15. However, this external dimension of the seal mechanism 21 is dimensioned so that it is bigger by at least twice the wall thickness 13 of the housing container 5 than the internal dimension 14 of the internal cross-section and thus the interior 10. Since the projection 30 constituting the coupling part 25 has an internal orifice width which essentially corresponds to the internal dimension 14 of the housing container 5 at its top end 6, the shoulder 32 is very securely retained in the cap 20 and an efficient seal is formed between the interior 10 of the housing container 5 and the atmosphere surrounding the housing unit 1.

Above all, the seal afforded by the closure device 9 for the open terminal end of the housing unit 1 can be improved if an external diameter of the seal mechanism 21 in the region of its seal surface 33 in the relaxed state outside the housing container 5 is bigger than the internal dimension 14 of the housing container 5 in the region facing the seal mechanism 21.

In the relaxed, non-mounted state, a longitudinal or vertical extension of the shoulder 32 of the seal mechanism 21 in the direction of the longitudinal axis 15 is bigger than a distance of a groove-shaped recess between the two projections 29, 30 and optionally minus a thickness of the retaining ring 31. Due to the dimensional differences between the groove-shaped recess and the longitudinal dimensions of the shoulder 32 and the thickness of the retaining ring 31 in the direction of the longitudinal axis 15 described above, the shoulder 32 is pre-tensioned between the two projections 29, 30. This simultaneously affords a seal and pre-tensions the seal mechanism 21 with respect to the cap 20 and also optionally produces a secure seating of the retaining ring 31 and a tight abutment of the two end faces of the shoulder 32 in the region of the two projections 29, 30.

It is also of advantage if the cap case 23 is provided in the form of a truncated cylinder case or truncated cone case, as a result of which the cap case 23 will extend round into the region of the top terminal end of the housing container 5.

It may also be of advantage to provide at least two guide projections 37, 38 in the region of the open terminal end 19 of the housing container 5, which project out from the external circumference of the cylindrical housing container 5. However, any number of guide projections 37, 38 may be provided, in which case they co-operate with guide webs 39, 40 disposed on an internal face of the cap 20 facing the housing container 5 and projecting from its surface in the direction towards the longitudinal axis 15. This being the case, the number and the, for example uniform, angular distribution of the guide webs 39, 40 around the circumference will depend on the number of guide projections 37, 38 provided on the housing container 5. These guide projections 37, 38 co-operate with the guide webs 39, 40 disposed on the internal face of the cap case 23, as a result of which, when the cap 20 is pushed on in the direction of the longitudinal axis 15 of the housing container 5 into the open terminal end thereof and turned accordingly in a clockwise direction, the guide webs 39, 40 run against the guide projections 37, 38 and due to the combined rotating and translating movement caused by the guiding action of the guide webs 39, 40 along the guide projections 37, 38, the seal mechanism 21 with its seal surface 33 can be inserted or pushed into the interior 10 of the housing container 5.

Also illustrated in the interior 10 of the housing container 5 is the separating device 11 with its base body 41, which has a surface directed towards the housing container 5 beyond which a seal mechanism 42 may optionally extend. This seal mechanism 42 may be provided in the form of one or more sealing lips, for example. It is of advantage if the material used for the base body 41 is of an elastically rebounding and deformable type, such as a silicone rubber, pharmaceutical rubber, bromobutyl rubber, rubber, a gel or an elastomeric plastic. Irrespective of the above, however, a plastic may be selected which is fluid-tight, in particular watertight, and optionally impermeable to gas, and for example may be selected from the group comprising polyethylene terephthalate (PET), polypropylene (PP), polyethylene (PE), polystyrene (PS), high-density polyethylene (PE-HD), acrylonitrile butadiene styrene copolymers (ABS) or similar or a combination thereof. Likewise, various additives may also be incorporated in the material in order to adapt exactly to the pre-definable density. A density should be between 1.02 g/cm³ and 1.07 g/cm³, preferably between 1.04 g/cm³ and 1.05 g/cm³.

Illustrated between the closure device 9 and the base body 41 of the separating device 11 in a very simplified format is another part, namely an independent, separate filter element 43, which is disposed directly adjacent to the sealing stopper 22 of the seal mechanism 21. It is of advantage if the filter element 43 has a three-dimensional shape on the side of the base body 41 and facing the sealing stopper complementing it. A detailed description of this and the different options for providing it will be described in connection with the explanations of the different embodiments of the base body 41.

However, it may also be of advantage if the material of the base body 41 is fluid-tight and is also a plastic which optionally has additives or fillers incorporated, such as a thermosetting plastic, a glass-clear polystyrene or similar. The base body 41 should also be permeable to gas, thereby permitting gases to pass through virtually unhindered at least for a period of 48 or 72 hours. It may also be of advantage if the total weight of the base body 41 and/or the separating device 11 and/or the filter element 43 is variable, as a result of which it will be possible to adapt the entire separating device 11 and/or its base body 41 and/or its filter element 43 exactly to different media 3, 4 of the mixture to be separated 2. In order to produce an exact physical separation of the two media 3, 4 from the mixture 2 during the centrifugation process, the specific weight for the density of the material of the base body 41 may be lower than the higher specific weight or density of one medium 3,4 to be separated by the separating device 11 on the one hand and higher than the lighter specific weight or density of another medium 3, 4 to be separated by the separating device.

However, it would also be possible for the base body 41 to serve as a ballast or tensioning means for the filter element 43, in which case a higher density could be selected compared with the constituents of the mixture 2 to be separated.

Depending on the mixture to be separated 2 into the different media 3, 4 or constituents, it may be of advantage if at least part-regions or the entire internal wall 19 of the interior 10 is provided with a coating, in order to assist the sliding movement of the separating device 11, in particular its base body 41 and/or filter element 43, during the separation process and/or to produce a chemical and/or physical effect on the mixture 2 or similar, as indicated in a part-region by broken lines. When the base body 41 is in the position in the housing unit 1 in the region of the open terminal end in which it is used, at least the internal wall 19 disposed between the separating device 11 and the oppositely lying end 7 may be provided with this coating, which is such that it can be detached from or dissolved off the internal wall 19 on contact with the mixture 2, and is applied at the same time as the separating device 11 is fitted, for example.

In the embodiment illustrated as an example here, the base body 41 of the separating device 11 has a flow passage 44 formed by a connection orifice at its center or in the region of the longitudinal axis 15, between mutually spaced end regions 45, 46 in the direction of the longitudinal axis 15, and in the end region 45 illustrated here a concave cut-out 47 may also be provided in addition, the shape of which is more or less adapted to or corresponds to the part of the seal mechanism 21 facing it, for example the sealing stopper 22 of the closure device 9. This being the case, it is of advantage if the cut-out 47 approximately has the shape of a funnel in its initial position, which will assist the flow of the mixture 2 introduced by the schematically illustrated cannula into the connection orifice or into the flow passage 44 in the base body 41.

As also schematically illustrated, a separate insert part 48 is inserted in or introduced into the connection orifice or flow passage 44. The design of the base body 41 and the insert part 48 will be described in more detail with reference to the next set of drawings.

The separating device 11 illustrated in FIGS. 2 and 3 is one possible and optionally independent embodiment and is shown in a simplified format on a larger scale. The same reference numbers are used for the same parts as those illustrated in FIG. 1. For the sake of simplicity, the filter element 43 has been left out of the diagram.

In the direction of the longitudinal axis 15, the base body 41 has the mutually spaced end regions 45, 46, which are spaced apart from one another by a distance 49 or height. In the first or top end region 45, the base body 41 has an external dimension 51 in a plane 50 oriented perpendicular to the longitudinal axis 15, which is bigger than another external dimension 52 in the end region 46 in another plane 50a likewise oriented perpendicular to the longitudinal axis 15 and extending parallel with the first plane 50. Since the cross-sections in the aforementioned planes 16, 17 of the housing container 5 or in the planes 50, 50a of the base body 41 are more or less circular, the separating device 11 can easily be inserted in the interior 10 of the housing container 5 irrespective of position. As a result, the base body 41 has the shape of a truncated cone in the region of its external surface, which has a cone angle 53 of between 0.1° and 3.0°, preferably between 0.6° and 0.8°. This cone angle 53 may correspond to the taper of the interior 10 of the housing container 5 between the two mutually spaced planes 16, 17 when the base body 41 is in the relaxed or non-deformed state.

It may be of advantage if the cone angle 53 of the base body 41 is slightly bigger than the taper of the interior 10, because this will reliably prevent the base body 41 from jamming in the transition region between the end region 46 and its external surface on the internal wall 19 of the housing container 5. Due to the materials selected for the housing container 5 and separating device 11, in particular its base body 41, it is possible to adapt accordingly to the elasticity behavior and as a result to the pressure or frictional force exerted by the external surface on the internal wall 19 of the housing container 11 and the associated sealing action.

The essential aspect here is that an internal circumference or an internal dimension 14 of the housing container 5 in the region of the first plane 16, 50 is the same as or smaller than an external circumference or an external dimension 51 of the base body 41 in its non-deformed state in the same plane 16, 50. This means that the external dimension 51 of the base body 41 in the plane 50 oriented perpendicular to the longitudinal axis 15 in its first end region 45 in the non-deformed state is the same as or bigger than the internal dimension 14 of the housing container 5 at its first open end 6 in the same plane 16.

FIG. 3 shows a view of the base body 41 from above, together with a gap 54 extending in it between the two end regions 45, 46 and widening in a conical shape starting from its center or longitudinal axis 15 through to its external surface. As described above, because of the size of the circumference or external dimension 51 selected for the base body 41 by reference to the internal dimension 14 of the housing container 5 in the plane 16 illustrated in FIG. 1 in conjunction with the gap 54, these components can easily be adapted to one another. With the same internal and external dimensions 14, 51, the external surface can be retained between the external surface and the internal wall 19 of the housing container 5 with low retaining forces in the region of the initial position.

If the external dimension 51 or external circumference of the base body 41 in the non-deformed or relaxed state is selected so that it is bigger than the internal dimension 14 in the initial position, a predefined retaining force can be obtained due to the elastic deformation and the associated pre-tensioning of the base body 41 in co-operation with the gap 54 between the external surface of the base body 41 and the internal wall of the housing container 5. Based on a selection of the pre-tensioning and differences in the dimensions, therefore, the displacement force which has to be applied in the direction of the longitudinal axis 15 due to the effect of the centrifugal force in order to produce a displacement or shift from the initial position in the direction of the operating position can be fixed. However, this also enables a sufficient retaining force for the operation of piercing the filter element 43 by the cannula to be influenced or fixed.

As also illustrated in FIG. 3, an arc length 55 of the gap 54 in the region of the external surface in the initial position in the housing container 5 and illustrated in FIG. 1 is the same as the circumferential difference between the internal circumference of the housing container 5 in the plane 16 oriented perpendicular to the longitudinal axis 15 in the region of the initial position and the internal circumference of the housing container 5 in the region of the operating position illustrated in FIG. 7, as will be illustrated and described below.

The gap 54 has these delimiting gap faces 56, 57, between which only a passage for one of the two media to be separated—the lighter medium 3 in the embodiment illustrated as an example here—would be provided during the pre-definable displacement from the initial position to the operating position. However, operating modes also occur in which the heavier medium 4 is not totally prevented from passing through. To this end, the filter element 43 briefly described above is provided in combination with the base body 41, which is disposed upstream of the flow passage 44 in the initial position and directly adjacent to the sealing stopper 22. To proceeding with filling, both the sealing stopper 22 and the filter element 43 must be pierced by the schematically illustrated cannula and the mixture 2 fed through the cannula then passes through the flow passage 44 into the interior 10.

As may also be seen by comparing FIGS. 2 and 3, the flow passage 44 or connection orifice briefly described above in connection with FIG. 1 is disposed in the base body 41, at its center or in the region of the longitudinal axis 15. This flow passage 44 permits the filling operation through the base body 41 of the separating device 11 into the interior 10 of the housing container 5. This flow passage 44 has a clearance width 58 in the portion facing the first end region 45 which is dimensioned so that the mixture 2 with which the interior 10 is to be filled can flow through unobstructed. As also illustrated, the flow passage 44 becomes wider, in particular assuming the shape of a truncated cone, starting from the first end region 45 with the clearance width 58 in the direction of the other end region 46t. As described above, the base body 41 may have the concave cut-out 47 in its first end region 45, which has a depth 59 in the region of the longitudinal axis 15, starting from the plane 50 in the direction of the longitudinal axis 15. The widening region of the flow passage 44 extends across a part-region of the distance 49 minus the depth 59 between the two end regions 45 and 46 or planes 50 and 50a.

Illustrated in the portion of the flow passage 44 which widens in a truncated cone shape is the insert part 48, which is disposed in a position closer to the portion of the end region 46. When the housing unit 1 is in its normal or usage position, the first end 6 is higher than the other end 7, and as a result of gravity or the gravitational force of the insert part 48, it is always in a position or disposition in the region close to the end region 46.

In order to prevent the insert part 48 from moving out of the flow passage 44 in the direction of the interior 10 of the housing container 5, a plurality of retaining mechanisms 60 is provided, as illustrated FIG. 4, which project into the flow passage 44 at the end region 46. The embodiment illustrated here is only one of many options, although it may be construed as an independent embodiment of the invention in its own right. In terms of its ability to move in the direction of the longitudinal axis 15, the insert part 48 is held in this portion of the base body 41, and the retaining mechanism 60 is designed so that a flow passage is always formed irrespective of the position of the insert part 48 and a flow is able to circulate through the flow passage 44. In the embodiment illustrated as an example in FIG. 4, several webs 61—in this instance three webs—are provided, disposed on the base body 41, in particular integrally formed on it. The fact of providing the webs 61 and their design makes allowance for the gap 54 described above, to enable the closing movement of the gap 54 to a position where the two mutually facing gap faces 56, 57 sit in contact, as will be explained in more detail below when the operating position is described. Accordingly, a flow passage which can be closed as and when necessary is formed by the flow passage 44 and/or the gap 54, which can be closed with at least one automatically acting valve arrangement. This may be achieved by a contact of the insert part 48 on the delimiting walls of the flow passage 44 or connection orifice and/or due to the contact of the two gap faces 56, 57 with one another.

The external dimension of the insert part 48 is indicated by broken lines in FIG. 4 and the simple flow option between the external surface of the insert part 48 and the flow passage 44 may also be seen. As a result, even when the insert part 48 is lying against the retaining mechanism 60, a flow connection is possible between the two mutually spaced end regions 45, 46 of the base body 41 through the flow passage 44.

It is of advantage if the clearance width 58 (see FIG. 2) of the flow passages 44 at the first end region 45 in the plane 50 oriented perpendicular to the longitudinal axis 15, when the base body 41 or separating device 11 is in the initial position, is the same as or smaller than the external dimension of the insert part 48, in particular the part of the insert part 48 facing the end region 45. As a result, the insert part 48 is also prevented from moving through or out of the flow passage 44 from the base body 41 here.

In the embodiment illustrated as an example here, the insert part 48 is provided in the form of a truncated cone and is dimensioned so that when the base body 41 is in the non-deformed state or in the position in which the separating device 11 is in the initial position prior to starting the centrifugation process, a displacement is possible across a part-region of a length 62 (see FIG. 2) inside the flow passage 44 in the direction of the longitudinal axis 15.

The base body 41 illustrated in FIGS. 2 and 3 has a more or less annular recess 63 between the external contact surface or face and the flow passage 44 in the direction of the longitudinal axis 15, which extends from the other end region 46 in the direction of the first end region 45. As a result, a casing part 64 is formed in the region of the external circumference of the base body 41. The disposition and design of the recess 63 will depend on the material chosen for the base body 41, the selected density as well as the associated weight and may be freely selected from one application to another application.

FIG. 5 illustrates another possible and also optionally independent embodiment of the retaining mechanism 60 for fitting in the flow passage 44 of the base body 41, the same reference numbers being used to denote the same parts as those used in connection with FIGS. 1 to 4.

In this example of an embodiment, the flow passage 44 for accommodating the insert part 48, which is not illustrated here, may also become wider in a truncated cone shape across a part-region of the distance 49, starting from the region 45 through to the other end region 46, as described in detail with reference to FIGS. 2 to 4. The flow passage 44 based on the design of the retaining mechanism 60 has a cross-section that is smaller than the design based on an orifice 65, and a wall part 66 is formed between the orifice 65 and the flow passage 44 in the end region 46 of the base body 41. Several ribs 67 or grooves 68 may be provided on the wall part 66 facing the first end region, projecting out from and/or recessed into it. As a result, complete closure of the orifice 65 can be prevented due to the abutment of the insert part 48 on the wall part 66. Due to the possibility of opting for a combined arrangement of ribs 67 and grooves 68, a greater flow volume can be obtained between the insert part 48 and the wall part 66 through to the orifice 65. Likewise, however, another option is an alternating arrangement around the circumference of the orifice 65 or flow passage 44 between the ribs 67 and grooves 68.

FIG. 6 illustrates a similar embodiment of the base body 41 with a view to illustrating the separating device 11, described above in connection with FIGS. 2 to 4, but unlike the latter, the insert part 48 here has a different three-dimensional shape.

In this example of an embodiment, the insert part 48 has the shape of a sphere, which is illustrated in a position directly adjacent to the end region 46. Here too, in order to provide the flow connection between the two end regions 45, 46 through the flow passage 44, it is necessary to provide a retaining mechanism 60 based one of the embodiments described above. For example, an arrangement of several webs 61 may be provided or the wall part 66 may be provided with ribs 67 and/or grooves 68 recessed into it.

FIGS. 7 and 8 illustrate the housing unit 1 with the separating device 11 contained in it, which in this instance comprises the base body 41 and the filter element 43, by means of which the mixture 2 contained in the interior 10 is physically split or separated into the two media 3, 4 as described with reference to FIG. 1 by applying centrifugal force, in particular by means of a centrifugation process. During this process, the lighter medium 3 is contained in the interior 10 between the separating device 11 and the first end 6 or closure device 9 and the other, heavier medium 4 is contained in the housing container 5 between the separating device 11 and the end 7, which in this instance is closed.

As described above, the gap 54 (see FIG. 3) in the first plane 16 between the gap faces 56, 57 has the arc length 55 in the region of the external surface or contact surface of the base body 41. Starting from the plane 16, the separating device 11 moves in the direction of the other end 7, as a result of which, due to the cone shape of the interior 10, there is a constant reduction in the internal circumference and after a displacement by a displacement path 69, the two gap faces 56, 57 constituting the gap 54 are moved into contact with one another. As a result, it is therefore possible, during the movement or displacement of the separating device 11, to fix a continual decrease in the arc length 55 (see FIG. 3) of the gap 54 starting from the initial position through to the operating position illustrated in FIG. 7, so that a tight contact is obtained between the two gap faces 56, 57, which preferably affords a seal.

Once contact is established, the one-way elastic adjustment to or reduction in the arc length 55 of the gap 54 ends and an internal dimension 70 or the internal circumference of the housing container 5 in the plane 71 extending in the region of the operating position and oriented perpendicular to the longitudinal axis 15 corresponds to an external dimension 72 or the circumference of the base body 41 when the gap 54 is in the closed position.

Due to the pre-definable cone shape of the interior 10 of the housing container 5 and the pre-selectable arc length 55 of the gap 54 in the base body 41, the displacement path 69 by which the separating device 11 can be moved starting from the initial position through to the operating position can be exactly predefined, as a result of which a mechanical blocking or locking or retaining action is assured inside the housing container 5. Due to the pre-definable displacement path 69, it is possible, to set the position and hence the associated position of the separating device for the operating position irrespective of the quantity with which it is filled, without constituents of the mixture 2, in particular the medium 4, getting into the space between the separating device 11 and the first end 6 or closure device 9. Preferably, this displacement path 69 lies more or less at the half-distance between the planes 16 and 17. Furthermore, a mechanical stop (positioning mechanism) is provided as a means of restricting the displacement path 69 inside the housing container 5, as will be described in more detail with reference to the subsequent drawings.

During the centrifugation process, the entire separating device 11 migrates along the internal wall 19 of the housing container 5 in the direction of the longitudinal axis 15 through to the operating position, and the medium 3 is able to pass through the gap 54 and through the filter element 43 into the space between the separating device 11 and the closure device 9 or first end 6. Moreover, what is in this instance the lighter medium 3 can also still pass through the passage 44 because the centrifugal forces acting on the insert part 48 cause it to be moved into the region of the retaining mechanism 60. It is of advantage if the density of the insert part 48 has a value which is lower than the heavier of the two media 3, 4 and higher than that of the lighter medium.

As an alternative to the above, however, it is also possible to select the density of the insert part 48 so that it is lower than the density of the lighter medium—in this instance medium 3—because in any event, the insert part 48 floats on this medium and is moved in the flow passage 44 in the direction of the end region 45. Due to the complementary conical design, the flow passage 44 is sealed between the two mutually spaced end regions 45, 46. This is further assisted due to the reduction in the gap 54 up to the point at which the two gap faces 56, 57 make contact with one another, thereby slightly reducing the cross-section of the flow passage 44, which enables an additional clamping force to be generated between the base body 41 and the insert part 48 in the portion of the mutual contact surfaces. Consequently, the insert part 48 affords a seal in the operating position, in particular a fluid-tight seal, at the boundary walls of the widening portion of the flow passage 44.

The material or substance used for the insert part 48 is preferably a plastic, which is fluid-tight, in particular watertight, and it may optionally also be impermeable to gas, and may be selected from the group comprising polyethylene terephthalate (PET), polypropylene (PP), polyethylene (PE), high-density polyethylene (PE-HD), acrylonitrile butadiene styrene copolymers (ABS), polystyrene (PS) or similar or a combination of these. Likewise, however, various additives may be added to the material in order to adapt exactly to the pre-definable density. A density should be between 1.02 g/cm³ and 1.07 g/cm³, preferably between 1.04 g/cm³ and 1.05 g/cm³. It is also of advantage if the density of the insert part 48 is slightly higher than the density of the base body 41 because a flow through the flow passage 44 between the two part-chambers of the interior 10 of the housing container 5 separated by the separating device 11 is always possible until shortly before the operating position is reached.

It has also proved to be of advantage if at least certain regions of the base body 41 and/or the insert part 48 are provided with a coating, such as a silicone layer, for example, because this means that no blood cells will be left adhered to these components during the centrifugation process, thereby preventing any contamination of the separated medium 3 between the closure device 9 and the separating device 11.

The housing unit 1 as a whole can be assembled as follows.

The separating device 11, which in this instance comprises the base body 41 and the filter element 43, is inserted in the prepared housing container 5 through its open end 6, after which the interior 10 of the housing container 5 is reduced to a pressure below atmospheric pressure, in which respect it is of advantage to evacuate the entire area surrounding the housing container 5 to this negative pressure, and the closure device 9 is then inserted in the open end 6 of the housing container 5 affording a seal in order to preserve this negative pressure. By providing the gap 54 in the base body 41 as described above and due to the corresponding design of the filter element 43, once the latter has been inserted in the interior 10, it can still be evacuated to the desired negative pressure and only then is the sealing closure device 9 fitted on or inserted in the housing container 5 at its open terminal end in order to maintain the negative pressure.

A density of the base body 41 may be between 1.04 g/cm3 and 1.05 g/cm3 and that of the insert part 48 between 1.06 g/cm3 and 1.07 g/cm3. After overcoming a certain displacement path, the end region 46 of the base body 41 reaches the surface of the mixture 2, and because of the prevailing centrifugal forces, the media 3, 4 are already separated due to their different density values. Accordingly, the mixture, such as full blood for example, has a density of between 1.05 g/cm3 and 1.06 g/cm3. The density of serum or plasma is between 1.02 g/cm3 and 1.03 g/cm3 and that of the cellular constituents is approximately 1.08 g/cm3.

FIGS. 9 to 11 illustrate another housing unit 1 for a mixture 2 of the type described above, the same reference numbers being used to denote the same parts as those used in respect of FIGS. 1 to 8 described above. To avoid unnecessary repetition, reference may be made to the description of FIGS. 1 to 8 given above. The detailed description of the schematically illustrated filter element 43 will be given with reference to the subsequent drawings.

The housing unit 1 again comprises at least the housing container 5, with the interior 10 or a housing chamber with the internal wall 19 bounding or surrounding it. The housing container 5 also has two ends 6,7 spaced apart from one another in the direction of its longitudinal axis 15, at least one of which is provided with an orifice. In the region of the internal wall 19, an internal dimension 14 of the interior 10 in the region of the first end 6 in a plane 16 oriented perpendicular to the longitudinal axis 15 is bigger than the internal dimension 18 in the region of the other end 7 in the plane 17 parallel with it in the same spatial direction, as a result of which the dimension of the interior 10 tapers in a conical or cone-shaped arrangement. Provided on at least one of the open ends 6, 7 is a closure device 9 to close the housing container 5 as and when necessary, which can also be opened as and when necessary, although this is not illustrated here.

The separating device 11 is inserted in the housing chamber or interior 10, which, depending on the design of the sealing stopper of the closure device on the internal wall 19, is disposed, starting from what is here the open end in the direction of the other end 7, at a pre-definable distance from the open terminal end. The separating device 11 is bounded by the two mutually spaced end regions 45, 46 in the direction of the longitudinal axis 15. Disposed between these mutually spaced end regions 45, 46 is at least one flow passage 44 through which a flow can pass. At least one seal mechanism 73 is also disposed between the separating device 11 and the housing container 5, in particular its internal wall 19.

In the embodiment illustrated as an example here, the separating device 11 comprises at least two base bodies 74 or components, which cam be pressed against at least certain regions of the internal wall 19 of the housing container 5 by means of at least one pressing element 75 in the initial position.

The two base bodies 74 constituting the separating device 11 in this instance respectively form an approximately semicircular surface as viewed in the direction of the longitudinal axis 15, and in the operating position, in other words in the sealing position disposed between the mutually facing regions of the base bodies 74, the flow passage 44 is closed and sealed, in particular fluid-tight. This closed position of the flow passage 44 may be adapted in terms of its size by the reduction described above, starting from the bigger internal dimension 14 of the interior 10 through to the smaller internal dimension 18 in the region of the other end 7, so that when the separating device 11 has moved, starting from the initial position or starting position as far as its operating position, it is fixedly positioned there, without the heavier medium being undesirably able to pass through to the lighter medium once the centrifugation process has ended.

The pressing element 74 disposed between the base bodies 74 in this instance causes a pressure force directed radially in the direction of the internal wall 19 to be applied to the two base bodies 74, as a result of which the seal mechanism 73 is moved into contact with the internal wall 19, at least around certain regions of the circumference, in the initial position already.

In the initial position, the interior 10 disposed between the separating device 11 and the other end 7 can be evacuated through the flow passage 44. After evacuation, the closure device 9, in particular the sealing stopper 22, is then inserted in the interior 10 of the housing container 5 and thus stored in this state. This housing unit 1 is now ready for receiving body fluids, tissue pieces or tissue cultures, for example, in particular blood, and the sealing stopper 22, which is not illustrated here, as well as the filter element 43 can be pierced by means of a cannula or needle and the housing unit 1 can be filled due to the negative pressure in the interior 10.

The internal dimension 14 or an internal circumference of a casing line of the interior 10 in the first plane 16 is bigger than an external dimension 76 or external circumference of a casing line of the base body or bodies 74 in its or their operating position and in the same spatial direction. This ensures that in the initial position, mixture to be introduced into the interior 10 is able to pass through the flow passage 44. After filling, the centrifugation process described above is run and the mixture 2 is separated into the two media 3, 4. To this end, the flow passage 44 is established between the ends 6,7 of the housing container 5 in the region of the separating device 11 in the initial position. Due to the centrifugal force acting on the separating device 11, the separating device 11 moves, starting from the initial position as far as the operating position spaced at a distance apart, where an internal dimension 77 or an internal circumference of a casing line of the interior 10 is the same as or smaller than the external circumference of a casing line of the base body or bodies 74 or the external dimension 76 in the same position.

The base body or bodies 74 of the separating device 11 seal the flow passage or passages 44 automatically in the operating position, acting as a control curve due to the reduced dimension of the interior 10. This being the case, the reduction in the internal dimension 14 through to the internal dimension 77 in the region of the operating position may be constant or continuous. However, it would also be possible for a part-portion of the distance between the initial position and the operating position, starting from the initial position, to be cylindrical and the other part-portion to taper in a conical or cone-shaped arrangement.

In order to produce a reliable movement whilst the centrifugal force is being applied to the separating device 11, its density will depend on the density values of the individual media 3, 4 to be separated. If the mixture 2 is blood, the density of the separating device as a whole should be selected so that it is greater than 1.05 g/cm³. Depending on the level selected for the centrifugal force for the centrifugation process, the separating device 11 may also have a density of between 1.5 g/cm³ and 3.5 g/cm³, preferably between 2.0 g/cm³ and 2.5 g/cm³.

As may be seen more clearly from FIGS. 10 and 11, in the embodiment illustrated as an example here, in addition to the filter element 43, not illustrated here, the separating device 11 comprises two base bodies 74 and has pressing elements 75, preferably disposed centrally between them. Depending on the size of the external dimension 76 of the separating device 11, however, it may also have several of these base bodies 74. The essential aspect, however, is that the base bodies 74 always move at the same relative displacement speed by reference to the housing container 5 during their entire movement relative to the housing container 5, so that a common movement is effected during the centrifugation process and a sealing, in particular fluid-tight closure is also ensured between the two mutually separated part-chambers of the interior 10 in the housing container 5 and is so also in the operating position.

In order to produce as uniform as possible a pressure force, a pressing element 75 is provided for each of the base bodies 74 of the separating device 11, extending through the longitudinal axis 15 and on either side of a plane of symmetry 78 perpendicular to the flow passage 44 and thus between the mutually facing regions of the base bodies 74. In order to ensure that the base bodies 74 assume a sealing position in their operating position, the base bodies 74 constituting the separating device 11 can be moved relative to one another in a plane oriented perpendicular to the longitudinal axis 15, as a result of which they can always assume the same relative position with respect to one another and relative to the housing container 5 and can also be moved simultaneously.

By providing the pressing element or elements 75, the base bodies 74 of the separating device 11 can be held positioned in their relative position and thus also joined to one another as they move. The pressing elements 75 are advantageously disposed symmetrically with respect to the longitudinal axis 15 and may be formed by interconnected resilient webs 79 which are V-shaped as viewed in the direction of the longitudinal axis 15 and which converge with one another as viewed in the direction towards the longitudinal axis 15, for example. The base body 74 or base bodies 74 and the pressing element 75 or pressing elements 75 are preferably made from the same type of material as one another, in which case the separating device 11 may be manufactured in a single production process, for example in an injection mould by an injection molding process.

In order to obtain the sealing contact between the mutually facing regions of the base bodies 74, an appropriate cut-out 80 may be provided as indicated by broken lines in FIG. 10. As a result, the individual resilient webs 79 snap into the cut-out or cut-outs 80 during the movement from the initial position into the operating position, thereby permitting a flat abutment in order to seal the flow passage 44 between the base bodies 74. The resilient webs 79 constituting the pressing element 75 are joined to one another at the mutually facing end regions and are joined to the base bodies 74 in displacement at the end regions remote therefrom.

Due to the V-shaped design, a pressing force is generated by the pressing elements 75 and expended in the direction remote from the flow passage 44, the purpose of which is to permit a flow through the flow passage 44 until the base bodies 74 lie one against the other in the region of the mutually facing regions. This is necessary for filling the interior 10 on the one hand and for allowing preferably one of the media to be separated to pass through during the centrifugation process.

The mutually facing regions of the base bodies 74 preferably form mutually abutting, preferably flat seal surfaces 81 in the end region 45. In addition, however, it is also possible to provide a seal arrangement 82 between the base bodies 74 of the separating device 11, in the region of the end region 45 facing the first end 6 of the housing container 5, in order to seal the flow passage or passages 44. This seal arrangement 82 is indicated by broken lines in FIG. 11 in the region of the seal surfaces 81 and may be of various different designs. For example, it might comprise sealing lips engaging in one another or overlapping or flap seals, etc.

The seal mechanism 73 disposed between the separating device 11 and the internal wall 19 of the interior 10 should be disposed in the region of the end region 45 facing the first end 6 of the housing container 5 so that the mixture 2 is already prevented from collecting at the top end of the separating device 11 between the base bodies 74 and the internal wall 19, which would ultimately lead to subsequent mixing of the already separated media. This would be the case if, for example, the seal mechanism 73 were disposed at a distance apart from the first end region 45 in the direction of the other end region 46, in which case both constituents of the mixture would be able to penetrate this space during filling, which can no longer be emptied during the entire centrifugation process and can also not be separated, which would mean that at least part-quantities of both constituents would then remain in the interior 10 between the separating device 11 and the closure device 9, leading to contamination of what in this instance is the lighter medium.

The seal mechanism 73 is preferably provided in the form of at least one sealing lip 83 extending around the external circumference of the base bodies 74, which projects radially outwards from the base bodies 74 in the direction away from the longitudinal axis 15. Due to the fact that the sealing lip 83 is elastically deformable to a certain degree, certain manufacturing tolerances, in particular differences in diameter, can be compensated between the base bodies 74 and the housing container 5. The essential aspect is that when the sealing lip 83 is in the operating position, the region between the separating device 11 and the internal wall 19 of the interior 10 is completely sealed, in particular fluid-tight.

The base body or bodies 74 may be disposed between the mutually spaced end regions 45, 46, for example if opting for two base bodies 74 provided as half-cylinders in each case, with the sealing lip 83 projecting around their external circumference.

As an alternative to this however, it would also be possible for the base bodies 74 to be provided in the form of a portion of a hollow cylinder or hollow truncated cone in a region cooperating with the internal wall 19 of the housing container 5, in which case savings can be made on material. If only a hollow cylinder or hollow truncated cone is used, care must be taken to ensure that the seal surfaces 81 are provided between the base bodies 74 to ensure that the flow passage 44 is automatically sealed in the mutually abutting position.

As described above, the sealing lip 83 projects out from the base bodies 74, which therefore have a smaller external dimension than the internal wall 19 bounding the interior 10 across the entire displacement path. To prevent the separating device 11 from moving out of line or tilting during the displacement, it is of advantage to provide several support elements 85 on the base bodies 74 projecting out from their external circumference and an external surface 84 in the direction facing away from the longitudinal axis 15. These support elements 85 are preferably distributed symmetrically with respect to the longitudinal axis 15 around the external circumference on the external surface 84 and may be webs oriented parallel with the longitudinal axis 15. However, these support elements 85 may also be provided in the form of nubs, spherical projections, etc., projecting out from the external surface 84, which may be distributed in any pattern around the external surface 84.

In order to improve flow conditions between the two mutually spaced end regions 45, 46 and avoid dead volumes, the base bodies 74 may be provided with a baffle surface 87 in the region of the first end region 45 facing the first end of the housing container 5, which is preferably formed by cone portions 86 tapering in the direction towards the longitudinal axis 15 and towards the other end region 46. It is also of advantage if the base bodies 74 have a flow surface 88 in the region of the second end region 46 facing the other end 7 of the housing container 5 extending at an angle in the direction towards the longitudinal axis 15 and towards the first end region 45.

This enables the mixture to flow unobstructed into the interior 10 in the direction of the other end 7 of the housing container 5 during filling, and also to be guided by the peripheral regions, in other words from the region of the internal walls 19 in the direction of the flow passage 44. In addition, the inclined flow surfaces 88 prevent the lighter medium from passing through the flow passage 44 during the separation process and also prevent a dead volume from forming.

FIG. 12 illustrates another possible and optionally also independent embodiment of the separating device 11, the same reference numbers being used to denote the same parts as those used for the preceding FIGS. 1 to 11. As the separating device 11 illustrated here differs from the embodiment described with reference to FIGS. 9 to 11 on the basis of just a few details, reference may be made to the more detailed description given above. Here too, the filter element 44 has been left out of the drawing in order to provide a clearer illustration of the design of the base body 74.

Again with this embodiment, the separating device 11 comprises several, preferably two base bodies 74, which are joined so that they move together by means of the pressing element or elements 75 in the form of resilient webs 79. The two base bodies 74 are illustrated in a position in which the region of the flow passage 44 is sealed. To enable a better variation in the density of the separating device 11 as a whole in the embodiment illustrated as an example here, the base body or bodies 74 of the separating device 11 are each provided in the form of a support body 89 and the seal mechanism 73 and/or seal arrangement 82 on disposed on it or them. In this respect, it is of advantage if the materials from which the support body 89 and seal mechanism 73 and/or seal arrangement 82 are made are different from one another.

The support body 89 should have a higher density than the seal mechanism 73 and/or seal arrangement 82 and optionally should have a higher modulus of elasticity. This will mean that with a support body 89 of the same volume and using a material with a higher density, a higher weight can be obtained, which makes it possible to effect the displacement by applying a lower centrifugal force.

The seal mechanism 73 and seal arrangement 82 may be made from a silicone rubber, pharmaceutical rubber, bromobutyl rubber, rubber, a gel, a thermoplastic elastomer (TPE), thermoplastic polyurethane (TPU) or some other elastomeric plastic, and the material for the support body 89 may be selected from the group comprising polyethylene terephthalate (PET), polypropylene (PP), polyethylene (PE), polystyrene (PS), high-density polyethylene (PE-HD), acrylonitrile butadiene styrene copolymers (ABS), thermoplastic elastomers (TPE), thermoplastic polyurethane (TPU), ultra-high molecular polyethylene with a very high molar weight (PE-UHMW), polycarbonate (PC), polyamide (PA), polyoxymethylene (POM) or some other thermoplastic plastic and optionally a combination of these. The seal arrangement 82 may but need not necessarily be provided. The materials used for the seal mechanism 73 and seal arrangement 82 between the support bodies 89 may naturally also be different from one another. The sealing lips 83 constituting the seal mechanism 73 project out from the external surface 84 towards the side remote from the longitudinal axis 15 in order to produce a better seal and, as described in detail above, produce a sealed closure between the separating device 11 and the internal wall 19 when the centrifugation process is terminated. The other seal of the flow passage 44 between the base bodies 74, in particular the support bodies 89, is obtained due to the seal arrangement 82 co-operating with the first end region 45, which is illustrated in a simplified format in the form of seal strips. This seal arrangement 82 may be of various types and may be designed to provide the sealing abutment of the mutually facing regions of the base bodies 74 in the region of the flow passage 44.

Again, the pressing element or elements 75 is or are provided between the base bodies 74, which, for the sake of simplicity, are illustrated in the form of the mutually abutting resilient webs 79. However, the pressing element 75 could naturally also be provided in any other form, although care must be taken to ensure that a sufficient pressing force opposes the individual base bodies 74 on the one hand and a sealed closure of the flow passage 44 is obtained in the operating position on the other hand.

As also illustrated in this drawing, to provide a stabilized position in the region of the external surface 84 during the displacement, the latter may be provided with projecting support elements 85 in at least certain regions, for example in the form of elongate webs or ribs or spherical shoulders distributed around the circumference to provide support on the internal wall 19. The degree by which these support elements 85 project out from the external surface 84 is dimensioned so that they lie against the internal wall 19 during the entire displacement through to the point at which the operating position is reached, and the seal mechanism 73, in particular the sealing lip 83, also projects outwards in the direction towards the internal wall 19 beyond the casing line by the distance of the support elements 85. Due to the elasticity of the sealing lips 83, they are deformed around the support elements 85 in the region projecting beyond the casing line towards the side remote from the separating device 11. Depending on the degree of the projection, the displacement force which must be applied in order to obtain the displacement from the initial position through to the operating position can also be fixed.

If a continuous web is used as the support element 85, care must be taken to ensure that it is always separate from the sealing lip 83 to ensure an unobstructed deformation of the sealing lip 83 to produce a sealing abutment on the internal wall 19. An external casing line in the region of the support elements 85 is therefore smaller than the external diameter of the sealing lips 83 of the seal mechanism 73 in the non-deformed state. Due to the projection of the sealing lips 83 beyond the casing line, support elements 85 cause the sealing lips 83 to be deformed to a certain degree in the initial position already, due to the pressing force expended by the pressing element or elements 75. The degree of deformation depends on the degree of the projection of the sealing lips 83 beyond the casing line around the support elements 85. The separating device 11 is securely seated in the region of the operating position due to the abutment of the individual support elements 85 on the internal wall 19 of the housing container 11 on the one hand and due to the deformed sealing lips 83 in the position affording a seal with the internal wall 19 on the other hand.

FIG. 13 illustrates different design options for the housing container 5 in a single drawing in simplified format, and these options may naturally be combined with one another. To provide a clearer illustration, the separating device 11 and the closure device 9 have been omitted from this drawing.

Illustrated in the region of the initial position adjacent to the end 6 of the housing container 5 are various embodiments of retaining mechanisms 90 for the separating device 11 to be inserted in the interior 10 or housing chamber. On the right-hand part of the drawing, the retaining mechanism 90 is shown in the form of at least one shoulder 91 projecting out from the circumference of the internal wall 19 in the direction towards the longitudinal axis and/or at least one web 92 projecting out from at least certain regions of the circumference of the internal wall 19 in the direction towards the longitudinal axis 15. Both the shoulder 91 and/or the web 92 may extend round only certain regions of the circumference or may also extend continuously around the entire circumference of the internal wall 19.

In the top left-hand region of FIG. 13, another embodiment of the retaining mechanism 90 is illustrated, this time in the form of a reduction in the internal dimension 14 of the interior 10. This reduction can be achieved due to the fact that, starting from the end 6 of the housing container 5, the latter has the normal wall thickness of the housing container 5 as far as the retaining mechanism 90, for example, and with effect from the retaining mechanism 90 towards the direction of the other end 7 it has a bigger wall thickness, and the increase in the wall thickness is achieved due to a stagger in the internal wall 19 in the direction towards the longitudinal axis 15. As an alternative to this, however, it would also be possible to select the wall thickness of the housing container 5 between the initial position and the other end 7 in the range of the usual wall thickness and only the wall thickness between the initial position and what is here the open end 6 of the housing container 5 is thinner.

Depending on the design of the retaining mechanism 90, a positioning of the separating device 11, in particular the base bodies 41, 74 to the point at which a pre-definable centrifugal force is reached when the retaining forces are overcome and the separating device 11 moves relative to the housing container 5 as far as the operating position can be predetermined.

In order to obtain a different fixed positioning or fixed relative position of the separating device 11 in the region of the initial position, the retaining mechanism 90 between the housing container 5 and the separating device 11 may be provided in the form of a groove-shaped recess, which extends around the internal circumference of the internal wall 19 and is recessed into it, although this is not illustrated.

In order to obtain a fixed positioning or relative fixed position of the separating device 11 in the region of the operating position, a positioning mechanism 93 may be provided between the housing container 5 and the separating device 11. This positioning mechanism 93 may be provided in the form of a reduction in the internal dimension 77 of the interior 10 for example, and by providing a stop surface 94 oriented more or less perpendicular to the longitudinal axis 15. Both the other end region 46 of the separating device 11 or its base body 74 or also the seal mechanism 73 disposed in the first end region 45, in particular the sealing lips 83, or also the filter element 43 may move into abutment with this stop surface 94. This results in a sealed, in particular fluid-tight, closure between the mutually separated media on termination of the centrifugation process and does so for a longer storage period.

The reduction in the interior 10 starting form the initial position through to the operating position is also provided on the housing container 5 illustrated here, as described above, and thus forms the control curve for the automatic closure of the flow passage or passages 44 in the region of the base body 41, 74.

The taper of the housing container 5 in its interior 10 between the two mutually spaced apart planes 16, 17 described above may be between 0.1° and 3.0°, preferably between 0.6° and 0.8°.

This housing unit 1 can be used not only with all those embodiments of the separating device 11 where the closure of the flow passage 44 or connection orifice operates on the principle of a reduction in the internal dimension of the interior 10 starting from the initial position through to the operating position, but also those with a totally cylindrical design of the internal wall 19.

FIGS. 14 and 15 illustrate another possible and optionally also independent embodiment of the base bodies 74 used to form the separating device 11 with the pressing element 75, the same reference numbers being used to denote the same parts as those described above in connection with FIGS. 1 to 13. Again, to avoid unnecessary repetition, reference may be made to the detailed description given above with reference to FIGS. 1 to 13. To retain clarity in the drawings, the filter element 44 is omitted from the diagram in order to illustrate the design of the base body 74 more clearly.

The separating device 11 again comprises the base bodies 74 with the flow passage 44 formed between the mutually facing components. The seal mechanism 73 is in turn disposed in the first end region 45 in the region of the external circumference of the base bodies 74 in order to seal the part-chambers of the interior 10 to be separated and may correspond to the embodiments described above with reference to FIGS. 9 to 12. The same also applies to the design of the baffle surface 87 provided in the form of a cone portion, which tapers from the peripheral regions in the direction towards the longitudinal axis 15 and opens into the flow passage 44.

Starting from the other end region 46 of the separating device 11, the base bodies 74 are each provided in the form of hollow cylinder segments 95—in this particular instance, two components extending more or less in a semicircle. Disposed in the region of the converging baffle surfaces 87, terminal wall parts 96 are provided which are joined to the hollow cylinder segments 95, extending in a plane oriented more or less perpendicular to the longitudinal axis 15.

The pressing element 75 is again provided in the form of interconnected resilient webs 79, which are disposed in the pattern of a parallelogram as viewed in the direction of the longitudinal axis 15. The resilient webs 79 belonging to the respective oppositely lying base bodies 74 are joined to one another in the region of the flow passages 44 and supported on the oppositely lying hollow cylinder segments 95 in a plane offset from the flow passage 44 by approximately 90°, optionally by means of retaining webs 97.

Due to the symmetrical layout of the resilient webs 79 with respect to the flow passage 44 and the fact that the resilient webs 79 are supported on the hollow cylinder segments 95 offset from them at a right angle, the base bodies 74 are pressed against the respective oppositely lying internal walls 19 of the housing container 5 more or less symmetrically with respect to the flow passage 44 during the entire time they are disposed inside the housing container 5.

FIGS. 16 and 17 illustrate another possible layout of the pressing element 75 for the base bodies 74 constituting the separating device 11, this embodiment of the base bodies 74 being the same as that described in connection with the preceding FIGS. 14 and 15. To avoid unnecessary repetition, reference may be made to this description. Again, the filter element 44 has been omitted from the diagram in order to provide a clearer illustration of the design of the base bodies 74.

The pressing element 75 is again disposed centrally between the hollow cylinder segments 95 with respect to the longitudinal axis 15 between the base bodies 74, and the resilient webs 79 have an elongate contour which curves as viewed in the direction of the longitudinal axis 15, and the requisite pressing force is applied to the base bodies 74 more or less in the direction perpendicular to the flow passage 44 due to the complementary curvature. Disposed in the region of the longitudinal axis 15 is what in this instance is a circular connecting part 98, and the mutually facing ends of the resilient webs 79 are joined to it in a plane oriented more or less perpendicular to the flow passage 44. The other ends of the arcuately curved resilient webs 79 are joined to the internal face of the hollow cylinder segments 95 in more or less the same plane as them.

In the case of the pressing elements 75 described above in connection with FIGS. 14 to 17, the resilient webs 79 are respectively joined exclusively to the hollow cylinder segments 95 only at oppositely lying regions by reference to the flow passage 44, so that the springing action can be transmitted to the base bodies 74 unhindered. A join between the resilient webs 79 and the terminal wall parts 96 must be avoided at all costs.

FIG. 18 illustrates a different option for the layout of the pressing elements 75 between the base bodies 74 in a simplified diagram, the same reference numbers being used to denote the same parts as those illustrated in the preceding drawings FIGS. 1 to 17. Again, the filter element 44 has been omitted from the diagram in order to provide a clearer illustration of the design of the base body 74.

In this example of an embodiment, the individual pressing elements 75 are provided in the form of helical springs for example, which are supported on the mutually facing regions of the base bodies 74. In order to accommodate them in the mutually facing wall parts of the base bodies 74 when the flow passage 44 is in the closed and sealing position, appropriate cut-outs 80 may be recessed into at least one of these surfaces.

To make assembly easier and facilitate the mutual retention of the individual base bodies 74 relative to one another, at least one guide part 99 extends in the region of the pressing elements 75 starting from at least one of the base bodies 74 in the direction of the oppositely lying base body 74 and engages in a retaining orifice 100 recessed into the other base body 74, as illustrated. It is also of advantage if a retaining projection 101 is provided on the end region of the guide part 99 projecting into the retaining orifice 100, which projects out from the guide part 99 in the radial direction in its external dimension. The retaining orifice 100 has a bigger dimension in the radial direction towards the guide part 99 in the region of the retaining projection 101 than in the region directly adjoining the flow passage 44. In this region, the retaining orifice 100 is more or less of the same dimension as the guide part 99. Due to elastic deformation, the retaining projection 101 which has a bigger diameter can be pushed into the first part of the retaining orifice 100 and then snaps into the retaining orifice 100 which is bigger in order to accommodate the retaining projection 101. Due to the cooperation with the pressing element 75, the two base bodies 74 are pressed apart from one another in the region of the flow passages 44, thereby affording a restriction and preventing the base bodies 74 from falling apart due to the co-operation between the retaining projection 101 and the smaller retaining orifice 100.

However, it would also be possible for the pressing element or elements 75 not to be disposed in the region of the guide parts 99 as illustrated here, but instead to provide a separate pressing element 75 on one of the base bodies 74, as indicated by broken lines.

This pressing element 75 has a curved, elongate contour and is provided in the form of a resilient web 79, which is joined to one of the base bodies 74 at one end region and extends in an arc in the region of the flow passages 44 in the direction of the oppositely lying base body 74 as viewed in the direction of the longitudinal axis 15. Providing the guide parts 99 and the co-operating retaining orifice 100 enables the base bodies 74 to be oriented with respect to one another, and the pressing mechanism is again disposed between the two mutually facing regions of the base bodies 74 but separately from the guide part 99 so as to maintain a mutual spacing and form the flow passage 44.

FIG. 19 illustrates another possible design of the housing unit 1, which in the embodiment illustrated as an example here comprises the housing container 5 and an inner container 102 inserted in its interior 10. To provide a clearer illustration, the closure device 9 as well as the separating device 11 have been omitted from this diagram.

This housing unit 1 may be used with all embodiments of the separating device 11 in which closure of the flow passage 44 or connection orifice operates on the principle of a reduction in the internal dimension of the interior 10 starting from the initial position through to its operating position.

The housing container 5 projects by means of what in this instance is its open end 6 beyond a terminal end 103 of the inner container 102 by a pre-definable distance, which may be selected so that the terminal end 103 forms the retaining mechanism 90 described above for the separating device 11 to be inserted in the interior 10. The internal dimension 14 in the region of the terminal end 103 as regards the design of the flow passage 44 or connection orifice in the region of the separating device 11 is selected so that with regard to the separating device 11 and its base bodies 41, 74, a passage is always free to enable the interior 10 to be filled with the mixture 2.

In the region of the operating position of the separating device 11, the inner container 102 has an internal dimension 76 which is smaller than the internal dimension 14 in the region of the terminal end 103. As a result, keeping to the same external dimensions for the housing container 5, it is possible to obtain different sizes for the interior 10 in the region of the inner container 102 for example, and at the same time, the choice adapting the internal dimensions 14, 76 and 18 relative to one another can be used to fix the position of the operating position of the separating device 11 relative to the housing container 5. The design of the mutually facing external and internal surfaces of the housing container 5 and inner container 102 as well as the choice of materials may be based on the disclosures made in patent specifications EP 0 735 921 B1, AT 402 365 B and U.S. Pat. No. 5,871,700 A.

FIG. 20 illustrates another possible and optionally independent embodiment of the separating device 11, comprising the base bodies 74. To avoid unnecessary repetition, reference may be made to the description given above in connection with FIGS. 1 to 19. The same reference numbers are used to denote the same parts. Again, the filter element 44 has been omitted from the diagram with a view to providing a clearer illustration of the design of the base bodies 74.

In the embodiment illustrated as an example here, the flow passage 44 is also disposed between mutually facing regions of the base bodies 74, and the seal mechanism 73 with the sealing lip 83 is again provided in the first end region 45, preferably extending around the circumference of the individual base bodies 74. The design of the baffle surface 87 in the first end region 45 may be based on the designs described in connection with FIGS. 9 to 18.

The two base bodies 74 are pivotably connected to one another by a hinge joint 104 in an end region of the flow passage 44, and this hinge joint 104 may simultaneously also constitute one of the support elements 85. Also distributed around the circumference are other support elements 85, schematically illustrated in the region of the external circumference. The design of the support elements 85 and the pitch around the circumference may be freely selected depending on requirements.

The hinge joint 104 may additionally also constitute the pressing element 75, whereby the base bodies 74 are always pressed against the internal wall 19 to form the flow passages 44 inside the housing unit 1 during its use.

In addition, however, it would also be possible to provide one or more additional pressing elements 75 in the end region of the flow passage 44 lying opposite the hinge joint 104, as indicated by broken lines. This enables an additional force to be directed onto the mutually facing base bodies 74 and hold the flow passage 44 open in the initial position to permit a flow until it reaches the sealing operating position.

The hinge joint 104 may be made from the same material as that used for the base bodies 74 or alternatively from a different material. This hinge joint 104 is preferably made in the same processing operation as that during which the base bodies 74 are produced, thereby obviating the need for subsequent joining operations to assemble the separating device 11. The amount of work involved in inserting the separating device 11 in the housing unit 1 is also reduced because although the separating device 11 may be made up of several components, it can be inserted in the interior 10 as a single unit.

What is essential as regards the embodiments described in connection with FIGS. 1 to 20 is that a taper of the housing container 5 or the inner container 102 in its interior 10 housing chamber between the two planes 16, 17 is approximately between 0.1° and 3.0°, preferably between 0.6° and 0.8°. Variations of plus/minus 10% are also possible. The housing container 5 and/or the inner container 102 and/or the base bodies 41, 74 and/or the seal mechanism 73 or seal arrangement 82 and/or the pressing element 75 may be made from a fluid-tight, in particular watertight plastic, which may optionally be impermeable to gases. This plastic is selected from the group comprising polyethylene terephthalate (PET), polypropylene (PP), polyethylene (PE), polystyrene (PS), high-density polyethylene (PE-HD), acrylonitrile butadiene styrene copolymers (ABS), thermoplastic elastomers (TPE), thermoplastic polyurethane (TPU), ultra-high molecular polyethylene with a very high molar mass (PE-UHMW), polycarbonate (PC), polyamide (PA), polyoxymethylene (POM), silicone rubber, pharmaceutical rubber, bromobutyl rubber, rubber, a gel or a combination of these.

The base body or bodies 74 are components preferably made from materials selected from the group comprising PE-UHMW, PC, PA, POM or other thermosetting plastics. The pressing element 75 may be made from the softer material, such as that specified for the seal mechanism 73 or seal arrangement 82, or alternatively from the same material as that used for the base body or its base bodies 74. It would also be possible to use any of the other materials specified in the description of the drawings given above.

The base body 74 or also only part-regions of it may be provided with a coating in at least certain regions, in which case the coating may be a silicone coating, for example. The interior 10 of the housing container 5 may be evacuated to a pressure that is lower than atmospheric pressure prior to fitting and closing the closure device 9.

FIG. 21 illustrates another possible and optionally independent embodiment of the separating device 11, comprising at least a filter element 43 and a base body 41. The same reference numbers are used to denote the same parts as those illustrated in the preceding FIGS. 1 to 20. To avoid unnecessary repetition, reference may be made to the more detailed description given above in connection with FIGS. 1 to 20.

This separating device 11 has the schematically illustrated base body 41 and at least one flow passage 44 extending between the end regions 45, 46, which has a flow connection to the filter element 43. The filter element 43 has terminal ends 105, 106 spaced apart in the direction of the longitudinal axis 15, and several passages 107 are provided in the filter element 43 extending between the ends 105, 106, as schematically illustrated. This being the case, the individual passages 107 each have a flow cross-section which is between the constituents of the substance to be separated from one another in terms of its size. The filter element 43 is disposed before the flow passage 44 in the filling direction, as illustrated by the cannula, and in the embodiment illustrated as an example here, extends radially across the entire cross-section of the interior 10.

The filter element 43 can be pierced by the schematically illustrated cannula in order to introduce the substance into the interior of the housing container 5, and in this operating mode an inlet orifice 108 is formed inside filter element 43 by the cannula. This inlet orifice 108 should be formed only due to the forcing of the material used to make the filter element 43, and an actual removal of the material or punching or cutting out of it by the cannula should be avoided at all costs. Once the cannula has been removed from the filter element 43, the inlet orifice 108 made by the cannula should be predominantly close again automatically of its own accord, and this feature is a special material property of the material used for the filter element 43. The end region 45 of the base body 41 facing the open end 6 of the housing container 5 is directly adjacent to and faces the terminal end 106 of the filter element 43, and the end region 45 is advantageously connected to or retained on the terminal end 106 of the filter element 43, at least in certain regions. This being the case, the filter element 43 may be fixedly connected to at least one of the base bodies 41, 74, and this may be done by a bonded or welded connection, for example by ultrasound welding and/or laser welding, for example. In addition or as an alternative, it would also be possible to use mechanical connecting means, such as force-fit or positive connections between the filter element 43 and the base body 41.

In this example of an embodiment, the base body 41 has a smaller external dimension 51 than the internal clearance cross-section of the housing container 5. As a result, the external surface of the base body 41 is spaced apart from the internal wall 19 in its position inserted in the interior 10. The seal arrangement 82 in the form of the sealing lips 83 already described above may be provided on the external circumference of the base body 41, which seals the gap between the internal wall 19 and the external surface of the base body 41 and thus divides the interior 10 into part-chambers disposed on either side of the separating device 11.

It may also be of advantage to provide one or more of the support elements 85 described above on the external circumference in order to stabilize the position of the base body 41. As an alternative, however, it would also be possible to dispose the seal arrangement 82 in the end region 46 facing the region of what is in this instance the closed end 7 of the housing container 5 instead of the support elements 85, in which case the sealing lip or seal arrangement 82 is simultaneously used as a support element 85.

Providing the seal arrangement 82 in the region of the external circumference of the base body 41 affords a passage through the base body 41 for a part of the mixture 2 or substance exclusively through the flow passage 44, and after flowing through the flow passage 44, it has a flow connection to the passages 107 provided in the filter element 43. In terms of size, these passages 107 have a flow cross-section, depending on the mixture to be separated, which lies between the constituents of the substance mixture to be separated. In order to obtain a reliable separation in each of the possible operating modes, the individual passages 107 in the filter element 43 are selected so that, in terms of their respective flow cross-section, a passage is afforded exclusively for a first part of the constituents of the substance to be separated. In the case of separating blood, this will be the lighter medium 3, namely serum or plasma. Based on the choice of respective flow cross-sections, the individual passages 107 prevent the other constituents of the substance to be separated from passing through. In this particular case, this will be the medium 4 containing the cellular constituents of the blood.

However, it would also be possible for the individual passages 107 in the filter element 43 to permit a passage for the first part of the constituents of the substance to be separated—in this particular case the lighter medium 3—such as serum or plasma for example, only during the time a centrifugal force is acting on the filter element 43 during the separation process. This results in an even more reliable separation in each of the individual operating modes. When the separating device 11 is moved from the initial position into the operating position, the individual passages 107 in the filter element 43 could be designed so that they are closed in the operating position. This can be obtained on the basis of an elastic type of filter element 43 in conjunction with the conical taper of the interior 10 between the two planes 16, 17 already described in detail above, in which case the internal wall 19 acts in the manner of a control curve radially compressing the filter element 43 so that the passages 107 automatically close once the operating position is reached. It is of advantage if the filter element 43 is made from an elastic material for this purpose. The possible examples of materials are listed below.

As described briefly above, after the operation of piercing the filter element 43 by the cannula to make the inlet orifice 108 in it, it is of advantage if the inlet orifice 108, once the cannula has been removed from the filter element, closes automatically due to the intrinsic nature of the material. This may be achieved on the basis of the choice of material, which may have elastic properties but also memory properties of the material.

As an alternative to this, however, it would also be possible to use an elastic rebounding material for the filter element 43, at least in the region provided specifically for the piercing operation. However, it would also be possible for the filter element 43 to have a different density, starting from its center 109 and extending to its peripheral region 110 which can applied against the housing container 5. This being the case, the density of the filter element 43 may be higher at its center 109 than the density in its peripheral region 110. As another alternative, however, the density of the filter element 43 could be lower at its center 109 than the density in the peripheral region 110.

As may also be seen from the drawing, the filter element 43 comprises a disc-shaped base body 111, which is bounded by the two terminal ends 105, 106 as view in the direction of the longitudinal axis 15, and by the peripheral region 110 in the region of its external circumference. The filter element 43 also projects out from the base body 41 towards the side remote from the longitudinal axis 15 in this embodiment illustrated as an example.

FIG. 22 illustrates a part-region of the housing unit 1 in the region of the closure device 9. The same reference numbers and component names are again used to denote the same parts as those used for the preceding FIGS. 1 to 21. To avoid unnecessary repetition, reference may be made to the more detailed description given in connection with FIGS. 1 to 21 above.

The separating device 11 in this instance comprises the base body or bodies 74 and the filter element 43 disposed before it in the filling direction.

The base body or bodies 74 incorporate the cone portion 86 in the portion facing the closure device 9, and a cone-shaped space is formed between the terminal end 106 facing the end region 45 of the base body 74 and the base body 74. In the region of the external circumference, the base bodies may be joined to the filter element 43, as already described in detail above. The schematically illustrated cannula in this instance pierces both the sealing stopper 22 and the filter element 43 in preparation for the filling operation and also extends through the flow passage 44. This being the case, the flow passage 44 may be of a size, in terms of its cross-section in the region of the initial position of the separating device 11 which is such that the cannula is able to pass through without any forcing or piercing movement. The base body or bodies 74 is or are secured relative to the housing container 5 in the initial position with a pre-definable retaining force, which is sufficient to keep the entire separating device 11 and optionally the base bodies 74 stationary during the operation of piercing the filter element 43 by the cannula. This prevents any sliding movement relative to the above-mentioned components during the piercing and filling process and the separating device is reliably held in the initial position at the start of the centrifugation process.

The filter element 43 has at least a base body 111, and in the embodiment illustrated as an example here, also has a projection 112 on it, projecting out from the base body 111. This projection 112 may project out from the base body 111 both towards the side remote from the longitudinal axis and towards one of the two mutually spaced terminal ends 105, 106. If the projection 112 on the base body 111 is disposed so that it projects in the peripheral region 110 in the direction towards the internal wall 19, the projection 112 may also be provided as an elastically deformable seal element in the region of the filter element 43. This being the case, it is of advantage if the at least one projection 112 of the filter element 43 is disposed extending round the circumference of the base body 111. The at least one projection 112 of the filter element 43 may then also constitute a sealing lip.

FIG. 23 illustrates another possible embodiment of the separating device 11, comprising the base bodies 74 and the filter element 43. The same reference numbers or component names are again used to denote the same parts as used in the preceding FIGS. 1 to 22. To avoid unnecessary repetition, reference may be made to the more detailed description given with respect to FIGS. 1 to 22 above.

In this example of an embodiment, the filter element 43 is provided with at least one additional seal element 113, such as an O-ring or similar in its peripheral region 110 which may face the internal wall 19 of the housing container 5. These additional seal elements 113 may be positioned either round the filter element or on the filter element, and during the displacement from the initial position to the operating position, the additional seal element 113 is moved as well. This being the case, the positive connection between the additional seal element 113 and the filter element 43 may be weaker than the retaining force between the filter element 43 and the base bodies 74, thereby preventing these two components from coming apart from one another during the displacement.

FIG. 24 illustrates another possible and optionally independent embodiment of the separating device 11, comprising the base bodies 74 and at least one filter element 43. The same reference numbers and component names are used for the same parts as in the preceding drawings. To avoid unnecessary repetition, reference may be made to the more detailed description given with reference to FIGS. 1 to 23 above.

In this example of an embodiment, the filter element 43 has at least one external dimension 114 in a plane oriented perpendicular to the longitudinal axis 15 which is the same as but preferably smaller than the internal clearance dimension 14 of the interior 10 in the region of the initial position. Due to the smaller external dimension 114, the flow passage 44 again establishes a flow connection with the passages 107 in the filter element 43 described above, and the terminal end 106 of the filter element 43 is supported in the region of the cone portion 86 of the base bodies 74.

An appropriate seal is provided between the base bodies 74 and the internal wall 19 of the housing container 5 by means of the seal mechanism 73 described above, in particular the sealing lips 83. In the case of a split design of the base bodies 74, care must be taken to ensure that a continuous seal is obtained relative to the internal wall 19 in the circumferentially extending peripheral region when the base bodies 74 are in the initial position, which is also continuous around the region of the mutually facing seal surfaces 81. To this end, however, additional sealing means may be provided in the region of the seal surfaces 81, although these are not illustrated here, which are elastically highly deformable and reliably prevent the media 3, 4 from flowing through. This ensures that the media 3, 4 can flow exclusively through the flow passage or passages 44 to the passages 107 in the filter element 43 during the separation process but also during storage of the housing unit 1. In this example of an embodiment, the sealing lip 83 surrounds the filter element in at least certain parts of its peripheral region 110, and the filter element 43 can also be retained by the sealing lip or lips 83 during the downward movement.

FIGS. 25 to 28 illustrate possible embodiments of the separating device 11, which may be construed as independent embodiments in their own right, comprising the base bodies 74 and at least one filter element 43. The same reference numbers or component names are again used for the same parts as those used for FIGS. 1 to 24 above. To avoid unnecessary repetition, reference may be made to the more detailed description of FIGS. 1 to 24 above.

FIG. 25 is a simplified diagram showing how the filter element with the base bodies 74 are attached and secured to one another by a bonding process, for example by means of a layer of adhesive. When choosing the adhesive system, allowance needs to be made for the materials used to make the base bodies 74 and the filter element 43 on the one hand and that it must be ensured that it is authorized for medical applications on the other hand.

FIG. 26 illustrates another option for this connection, namely ultrasound welding, whereby an energy-directing transmitter 115 is disposed on at least certain regions of one of the parts to be joined to one another—filter element 43 and base body 74. The energy can therefore be directed or bundled during the ultrasound welding process in order to produce a perfect connection between the base bodies 74 and the filter element 43.

FIG. 27 illustrates the fact that the base bodies 74 have at least one annular wall part 116 on the side facing the filter element 43 which is integrally formed and has a groove-shaped recess 117 for retaining the filter element 43. Accordingly, the filter element 43 is inserted in this groove-shaped recess 117. This recess 117 in the form of an annular groove may also be used as a means of compensating tolerances for the filter element 43, in which case the retaining forces inside the recess 117 during a downward movement must be stronger than the centrifugal forces acting on the filter element 43. This groove-shaped recess 117 may be made during an injection molding process, although, as an alternative, it would also be possible for the wall part 116 to follow a straight line initially and then mould appropriate retaining tabs or retaining projections by a heat process once the filter element 43 has been inserted.

FIG. 28 illustrates an embodiment similar to that shown in FIG. 27 but in the embodiment illustrated as an example in this instance, a circumferentially extending annular groove is not provided and instead, several snap hooks 118 are distributed around only certain regions of the circumference of the base body 74. The snap hooks 118 project out from the filter element 43 towards the side facing away from the base body 74 and the filter element 43 is therefore joined to the base body or bodies 74 to form the separating device 11. An appropriate seal is provided in the region of the external circumference of the base bodies 74 and the internal wall 19 and may be of the type corresponding to the embodiment described in connection with FIG. 24. The same also applies to the embodiment of the base body, which may be of the type described in connection with FIG. 27.

FIG. 29 illustrates another embodiment of the separating device 11, which may be construed as an independent embodiment in its own right, comprising the base bodies 74 and at least one filter element 43. The same reference numbers and component names are used for the same parts as those described with respect to the preceding FIGS. 1 to 28. To avoid unnecessary repetition, reference may be made to the more detailed description given with respect to FIGS. 1 to 28 above.

In this example of an embodiment, both the filter element 43 and the base bodies 74 are surrounded by a common housing 119 at least in the region directed towards the internal wall 19 of the housing container 5. It is of advantage if the housing 119 is of an elastically deformable design, at least in the radial direction with respect to the longitudinal axis 15, as a result of which allowance can be made for the reduction in the internal dimension 14 in the direction towards the smaller dimension 18 during the displacement starting from the initial position through to the operating position. This flexibility may be achieved on the basis of the density and/or pore size of the housing 119, for example. As a result, a seal is provided in the region of the external circumference of the separating device 11 between it and the internal wall 19 in all operating modes. It is also of advantage if the housing 19 is impermeable to the constituents to be separated from the substance mixture 2. This ensures that the mixture 2 or one of its media 3, 4 flows exclusively in the region of the flow passage 44.

FIGS. 30 to 33 illustrate possible embodiments of the separating device 11 which may optionally be construed as independent embodiments in their own right, comprising the base bodies 74 and at least one filter element 43. The same reference numbers and component names are used for the same parts as those described in connection with FIGS. 1 to 29 above. To avoid unnecessary repetition, reference may be made to the more detailed description of FIGS. 1 to 29 above.

FIG. 30 illustrates another possible embodiment of the retaining mechanism 90 between the separating device 11 and the housing container 5. In this instance, the position of the base body 74 is fixed by at least one but preferably several shoulders 91 projecting out from the internal wall 19 in the direction towards the longitudinal axis 15, which preferably engage in a circumferentially extending cut-out 120 of a complementary design in the initial position. The position of the entire separating device 11 is therefore fixed in its initial position, including during the operation of piercing the filter element 43 by the cannula. When the pre-definable centrifugal force is applied to the separating device 11 in order to separate the mixture 2 into the two media 3, 4 this retaining force or forces is or are overcome and the entire separating device 11 is moved from the initial position into the operating position, as described in more detail above.

In the embodiment of the retaining mechanism 90 illustrated in FIG. 31, a cut-out is provided both in the peripheral region of the base body 74 and in the internal wall 19, and an O-ring may be used as the retaining means in this instance, which produces a seal around the entire circumference of the base body 74 with respect to the internal wall 19. During the displacement from the initial position into the operating position, the sealing ring may migrate with the separating device 11 or alternatively may remain in the cut-out 120 provided in the housing container 5, in which case the separating device 11 moves into the operating position without this sealing ring.

In the embodiment of the retaining mechanism 90 illustrated in FIG. 32, it is disposed between the base bodies 74 of the separating device 11 and the sealing stopper 22 of the closure device 9. In this instance, the base bodies 74 have wall parts 121 directed towards the sealing stopper 24, which may be of a tubular design for example. The sealing stopper 22 has at least one but preferably several retaining shoulders 122 directed towards the base bodies 74 in its external peripheral region in the region of its seal surface 33, which engage with the wall parts 121 when the separating device 11 is in the initial position. The retaining shoulder 122 may also be provided in the form of a tubular component, however, with at least one but preferably several retaining lugs 123 projecting in the direction towards the longitudinal axis 15, which engages in a complementary cut-out 120 in the wall part 121. This again fixes the position of the separating device 11 in its initial position until the start of the movement into the operating position.

In the case of the embodiment illustrated in FIG. 33, the retaining mechanism 90 is assigned not to the base body 74 but to the filter element 43.

The design of the housing container 5 may be of the type described in connection with FIG. 13 where the container wall 12 has a differing thickness. With the abutment surface for the filter element 43 which is disposed at a right angle with respect to the longitudinal axis 15 in this instance, the filter element 43 sits on this step and is able to slide off it during the down-ward movement from the initial position into the operating position. The base bodies 74 may be additionally retained in the manner described in detail above in connection with the other drawings, between it and the housing container 5.

In all of the embodiments of the separating device 11 described above, at least one filter element 43 is provided in each case in the region remote from the interior 10 before the flow passage 44 in the filling direction, and it is possible for one of the media to be separated 3, 4 to flow between the terminal ends 105, 106 exclusively through the passages 107 extending in the filter element 43. An additional separation of the para-chambers in the interior 10 is provided in the operating position due to the fact that the flow passage 44 is able to close automatically when the base bodies 41, 44 are in the operating position. During the separation process and the displacement of the separating device 11 from the initial position into the operating position, the mixture 2 is physically separated due to the centrifugal force applied to it and as the separating device 11 sinks, the lighter medium 3 flows through the flow passage 44 and then on through the passages 107 in the filter element 43.

When the flow passage 44 is in the operating position and sealed by one of the various options described in detail above, the lighter medium 3 exclusively is disposed in the part-chamber of the interior 10 between the separating device 11 and the open end 6 or closure device 9. Depending on the filling volume and position of the operating position of the separating device 11 relative to the housing container 5, a mixture 2 of the two media 3, 4 may still be contained between the filter element 43 and a free space form in the base body 41, 73—for example in the cone portion 86. In this case, a flow of the lighter medium 3 out of the region of the separating device 11 through the filter element 43 into the part-chamber disposed between the filter element 43 and the closure device 9 must be prevented at all costs, in order to avoid any subsequent contamination of the medium 3. This being the case, it is of advantage if the passages 107 are closed or reduced in size to the degree that the lighter medium 3 is reliably prevented from flowing out of the free space or gap between the filter element 43 and the base bodies 41, 74 when the separating device is in the operating position.

It is also of advantage if the base body 41, 74 and the filter element 43 have a different density from one another, whereby for example the base body 41, 74 will have a higher density than the filter element 43. As an alternative however, it would also be possible for the base body 41, 74 and the filter element 43 to be of the same density.

The material used for the filter element 43 may be selected from the group comprising polyolefins, such as polyethylene or polypropylene, polyamide, polystyrene, polyether sulfane, polyester, thermoplastic elastomers (TPE, TPU), glass fibers, cellulose or compounds thereof, for example, such as mixed cellulose esters, cellulose acetate, cellulose nitrate, for example, or other natural fibers such as cotton fiber, or a combination of these. The density of the separating device 11 device as a whole should be greater than 1.05 g/cm³, preferably greater than 1.1 g/cm³.

The filter element 43 of the separating device 11 is pierced in the initial position and the piercing point of the filter element 43 should close automatically one the cannula is removed. It is of advantage if this piercing point in the filter element 43 is closed by the displacement of the separating device 11 into the operating position. Accordingly, the separating device 11 slides on the internal wall 19 from the initial position into the operating position, and the change in dimension, in particular the change in diameter, can be accommodated by the base body 41, 74 or by the filter element 43 or by both.

FIGS. 34 and 35 illustrate another possible and optionally independent embodiments of the separating device 11, comprising the base body 41 and at least one filter element 43, as well as the housing container 5. The same reference numbers and component names are used to denote the same parts as those described in respect of FIGS. 1 to 33 above. To avoid unnecessary repetition, reference may be made to the more detailed descriptions given with respect to FIGS. 1 to 33 above.

The separating device 11 illustrated in this instance comprises the base body 41 and the filter element 43, which is illustrated only in certain parts of the region of the longitudinal axis 15. To provide a circumferential seal for the filter element 43 inserted in the base body 41, the base body 41 has at least one but preferably several mutually spaced seal lugs 124 in the direction of the longitudinal axis 15. In order to fix the relative position of the filter element 43, a shoulder 125 is provided on the base body 41 on the side facing the closed end 7 am base body 41 projecting in the direction towards the longitudinal axis 15. The filter element 43 is retained and supported on it relative to the base body 41 as viewed in the direction of the longitudinal axis 15 as it moves farther in the direction towards the closed end 7.

As may also be seen from FIG. 34, the base body 41 is of an integral, approximately tubular design in this instance. Accordingly, due to its tubular design, the base body 41 surrounds the flow passage 44, which extends at least in certain regions between the two end regions 45, 46. The filter element 43 in this case is inserted in at least certain regions between the two end regions 45, 46 in the base body 41 and its flow passage 44. To accommodate the filter element 43, the base body 41 has an annular cut-out 126 on the side facing the first open end. The lateral boundary of the cut-out 126 is preferably of a cylindrical design by reference to the longitudinal axis 15, and the seal lug 124 projects out from this side wall.

To provide a circumferential seal for the separating device 11, in particular the base body 41, with respect to the internal wall 19 of the housing container 5, the base body 41 has the seal mechanism 42 in the form of a circumferentially extending sealing lip 127 in the region of its external circumference on the side facing what in this instance is the open end 6. The sealing lip 127 is designed so that during the relative movement from the initial position into the operating position, it is always in contact with the internal wall 19. In order to stabilize this longitudinal movement, at least one but preferably several guide elements 128 are provided on the base body 41 distributed around the circumference. These guide elements 128 may be provided in the form of webs or ribs and may be disposed in the region of the external circumference. The longitudinal contour is oriented parallel with the longitudinal axis 15. In this example of an embodiment, the guide elements 128 co-operate with the positioning mechanism 93 of the housing container 5 in the operating position.

As an alternative, however, it would also be possible for the guide elements 128 to be designed so that the sealing lip 127 moves into abutment with the positioning mechanism 93 of the housing container 5 after the relative movement from the initial position into the operating position, as will be explained below with reference to FIG. 36. This enables an axial fixing action to be produced in the direction of the longitudinal axis 15 relative to the housing container 5.

Another guide element 128 is indicated by broken lines in the region of the other end region 46 of the base body 41 on its external circumference, which may extend continuously around the circumference. The purpose of this guide element 128 is to stabilize the separating device 11 during the movement form the initial position into the operating position as well as its relative position with respect to the housing container 5 and produce a sealing contact on the internal wall 19. This also enables a smooth longitudinal movement in the direction of the longitudinal axis 15, without any jamming or tilting.

In order to obtain a full-surface mutual abutment of the terminal ends 105 of the filter element 43 facing the sealing stopper 22 on the seal surface 34 provided on or formed by the sealing stopper 22, the terminal end 105 of the filter element 43 is of a flat design, at least in the region of the longitudinal axis as well as in a plane oriented perpendicular to the longitudinal axis 15 and forms a contact surface 129.

In order to obtain a tight abutment of the contact surface 129 on the seal surface 34, it is also of a flat design at least in the region of the longitudinal axis 15 as well as in a plane oriented perpendicular to the longitudinal axis 15. As a result, the substance to be introduced into or the mixture to be introduced into the housing container 5 is prevented from prematurely leaving or being sucked out of this region where the sealing stopper 22 and filter element 43 merge during the operation of piercing the sealing stopper 22 by the cannula and in the direction towards filter element 43. This prevents contamination of the serum/plasma after the separation.

As illustrated in FIG. 35, the retaining mechanism 90 of the housing container 5 is also designed to accommodate the separating device 11 in the region of the open ends 6, and its dimensions may be adapted so that when the separating element 11 is supported on the retaining mechanism 90, the contact surface 129 of the filter element 43 moves into abutment with the sealing stopper 22, in particular its seal surface 34, and is pre-tensioned.

In order to produce a sufficient retaining force for the operation of piercing the separating device 11 relative to the housing container 5 on the one hand and to release the separating device 11 from the initial position in order to effect the relative movement into the operating position on the other hand, it is of advantage if the density of the separating device 11 is between 5.0 g/cm³ and 25.0 g/cm³, preferably between 10.0 g/cm³ and 15.0 g/cm³. This can be achieved by embedding at least one insert element 130 in the base body 41. This insert element 130 is of a higher density than the material of the base body and may be made from a metal material or similar, for example. This enables the total density of the separating device 11 to be increased significantly. The insert element 130 may be provided in the form of a tube or ring and may be injected into the base body 41 when it is being manufactured and thus embedded in it.

In the embodiment illustrated here, at least certain regions of the filter element 43 are surrounded by the base body 41, and the filter element 43 is advantageously completely enclosed by the base body 41 in its circumferential region facing the housing container 5. In order to produce a reliable abutment of the filter element 43 on the sealing stopper 22, the filter element 43 extends across the base body 41 on the side facing the sealing stopper 22 of the seal mechanism 21.

As an alternative, however, it would also be possible to provide at least one baffle element 131 for the filter element 43 in the region of its external circumference on the base body 41 at the terminal end 105 facing the sealing stopper 22. The purpose of this baffle element 131 is to deflect any impurities or residues which there might be in the in the direction towards the filter element 43 during the separation process, and to retain or fix the filter element 43 in its position relative to the base body 41. This prevents the base body 41 from inadvertently being released in the direction towards the open end 6. However, it would also be possible to integrally form or injection mould the base body 41 on the filter element 43 during the process of manufacturing the separating device 11, as is possible when running a two-component injection molding operation.

In order to prevent individual constituents of the substance or media 3, 4 of the mixture 2 from adhering, not only can a coating be provided on the base body 41, a coating can also be applied to the filter element 43 and/or introduced into the passages 107 of the filter element 43. For example, the coating might be a chemical substance, in particular selected from the group comprising silicone or silicone oil, or may be applied by nano-technology, in particular using nano-particles. This not only prevents or makes it more difficult for individual constituents of the substance to become adhered, it also facilitates or is conducive to the flow of constituents of the substance to be separated through the passages 107 by altering the surface properties or surface tensions.

The passages 107 in the filter element 43, illustrated in simplified format, should block any passage for the constituents or media 3, 4 to be separated in the initial position. In this respect, the passages 107 may have an internal dimension with a lower limit of 10 μm, preferably 15 mm, in particular 20 μm, and an upper limit of 25 μm, preferably 30 μm, in particular 50 μm. To enable the interior 10 to be reduced to a pressure below atmospheric pressure once the separating device 11 has been inserted and before closing with the closure device 9, the passages 107 may be designed so that air is able to pass through them, at least in the initial position.

It may be preferable for the housing container 5 illustrated in FIG. 35 for forming the housing unit 1 to be used in conjunction with the separating element 11 illustrated in FIG. 34. The interior 10 is bounded by the container wall 12 and the internal wall 19 is cylindrical by reference to the longitudinal axis 15, starting from the open end 6, in particular in the first plane 16, through to the positioning mechanism 93. As explained above, the positioning mechanism 93 may be provided in the form of a reduction in the internal dimension or cross-section of the interior 10, and for example in the form of a step, shoulder or similar. Adjoining the positioning mechanism 93, the internal wall 19 may taper in a conical arrangement as far as the other plane 17 in the region of the other end 7 by reference to the longitudinal axis 15, with a cone angle 132 of between 0.1° and 3.0°, preferably between 0.6° and 0.8°. As an alternative however, it would also be possible for the internal wall 19 of the housing container 5 to be cylindrical by reference to the longitudinal axis 15, starting from the positioning mechanism 93 through to the other plane 17 in the region of the other end 7. The external surface of the housing container 5 may be continuously cylindrical or may also taper in a conical arrangement along its longitudinal extension between the two ends 6, 7. Likewise, however, it might be that part portions are cylindrical and part portions taper in the manner of a truncated cone or cone.

As regards the different possible combinations of the design of the internal wall 19 described above, care should always be taken as regards the external surface of the housing container 5 that there is no drop below a minimum wall thickness of the container wall 12 across the entire longitudinal extension of the housing container 5. Keeping to this minimum wall thickness, depending on the material selected, in particular in the case of plastics, ensures that there is no undesirable drop in the pressure prevailing in the interior 10 end ensures that there can be no possible interaction between the interior 10 and the external atmosphere.

FIG. 36 illustrates another possible and optionally independent embodiment of the separating device 11, the same reference numbers and component names being used for the same parts as those described in connection with FIGS. 1 to 35 above. To avoid unnecessary repetition, reference may be made to the more detailed descriptions given with reference to FIGS. 1 to 35 above.

By contrast with the diagram shown in FIG. 34, the device 11 illustrated in this instance does not have the filter element 43 inserted in the base body 41, and instead has a separating element 133 with slightly different properties. Disposed in the separating element 133 are passages 134, which are schematically indicated by bold lines. The design of the separating device 11 and housing container 5 is preferably otherwise the same as that described in detail with reference to FIGS. 1 to 35.

Unlike the filter element 43 described above, the passages 134 provided in the separating element 133 are designed so that when the operating position is reached, the constituents of the substance to be separated are able to pass exclusively whilst a pressure force is being applied to the substance and separating device 11, in particular the separating element 133. When the operating position is reached and the pressure force or effect of the force is removed, the passages 134 prevent the constituents to be separated from flowing in both directions. In order to introduce the substance into the housing container 5, however, the separating element 133 is designed so that it can be pierced and once the cannula has been removed from the separating element 133, the inlet orifice 108 made by the cannula is designed to close again for the most part. During the separation process and whilst pressure is being applied and the movement is taking place, the flow passage or passages 44 of the base body 41 establish a flow connection with the passages 134 disposed in the separating element 133.

Since the density of the separating device 11 can also be selected so that it is significantly higher than the total density of the substance or the weight 2, the entire separating device 11 can be moved rapidly towards the positioning mechanism 93, for example, and a passage is then established for the heavier constituents during the centrifugation process, such as the cellular constituents in the case of blood, through the passages 134. During the rapid sinking or shifting movement towards the positioning mechanism 93, the separating element 133 is permeable to all the constituents of the substance to be separated because a pressure is exerted on the separating element 133. Due to the continuing effect of the centrifugal force on the substance to be separated, it is separated into its constituents, and the heavier constituents pass through the passages 134 if they are not yet located in the part-chamber between the separating device and the end 7 of the housing container 5. When the pressure is removed, in particular the centrifugal force, the separating device is already in the pre-definable position due to the co-operation with the positioning mechanism 93, and the constituents of the substance are prevented from flowing through the passages 134 in both directions. The relative position of the positioning mechanism 93 with respect to the housing container 5 and its holding capacity is selected so that if the housing unit 1 is used as a blood sample tube, there is sufficient space for the heavier or cellular constituents in the portion of the interior 10 disposed between the separating device 11 and the end 7 closed by the terminal wall 8. In the case of full blood, the cellular constituents account for a proportion by volume in a range of between 40% and 50%, usually 45%. For safety reasons, this part-chamber between the separating device 11 and the closed end 7 may be selected so that it is slightly bigger, to ensure that the cellular constituents can be safely contained. Accordingly, the volume capacity of the part-chamber formed between the separating device 11 and the other end 7 of the housing container 5 is selected so that it is at least the same as but preferably slightly bigger than the volume containing the heavier element, in other words the cellular constituents of the substance.

FIG. 37 illustrates another possible and optionally independent embodiment of the housing unit 1. The same reference numbers and component names are used for the same parts as those described in connection with FIGS. 1 to 36 above. To avoid unnecessary repetition, reference may be made to the more detailed description given in respect of FIGS. 1 to 36 above.

The housing unit 1 has at least the housing container 5, which is closed off at its open end 6 by means of the closure device 9. Inserted in the interior 10 is the separating device 11, but in this instance it is spaced at a distance from the sealing stopper 22 of the seal mechanism 21 in the direction of the longitudinal axis 15. This distance 135 or space is selected so that when the sealing stopper 22 is pierced by the schematically indicated cannula, the latter does not pierce the separating element 133, which is retained in the base body 41. The substance mixture 2 is introduced into the interior 10 due to the negative pressure prevailing in the interior 10. The substance mixture 2 is firstly introduced into the first part-chamber between the separating device 11 and the open end 6 and due to the prevailing pressure difference, the substance or mixture 2 is sucked between the part-chambers on either side of the separating device 11 as it is being introduced during the filling process, through the passages 134 into the other part-chamber. The separating element 133 is therefore permeable to the constituents of the substance as long as a pressure force is acting on the substance. This pressure force is applied to the substance due to the difference in pressure in the two part-chambers during the filling operation.

After the filling operation, the housing unit 1 is centrifuged in a manner known per se, and the constituents of the substance are separated on the basis of their density and the separating device 11 is automatically moved towards the pre-definable position in the region of the positioning mechanism 93 at the same time. After sufficient centrifugation, the centrifugation process is halted, and when the pressure force or pressure difference is removed, the passages 134 prevent any flow of the mutually separated constituents or media 3, 4 in both directions. In the embodiment described here, the separating element 133 is no longer subjected to the piercing stress of the cannula but it must be ensured that the substance to be introduced is able to pass almost completely through the separating element 133 and its passages 134 during filling. After the centrifugation process and once the separating device 11 has been positioned in the region of the positioning mechanism 93, however, a reliable separation of the mutually separated constituents is assured in both flow directions.

For the sake of good order, it should be pointed out that in order to provide a clearer understanding of the design of the separating device and housing unit, they and their constituent parts have been illustrated to a certain extent out of scale and/or on an enlarged scale and/or on a reduced scale.

The underlying objective of the independent solutions proposed by the invention may be found in the description.

Above all, the individual embodiments illustrated in FIGS. 1, 2, 3, 4; 5; 6; 7, 8; 9, 10, 11; 12; 13; 14, 15; 16, 17; 18; 19; 20; 21; 22; 23; 24; 25; 26; 27; 28; 29; 30; 31; 32; 33; 34; 35; 36; 37 may be construed as independent solutions proposed by the invention in their own right. The associated objectives and solutions may be found in the detailed descriptions of these drawings.

### LIST OF REFERENCE NUMBERS

1 Housing unit 31 Retaining ring
2 Mixture 32 Shoulder
3 Medium 33 Seal surface
4 Medium 34 Seal surface
5 Housing container 35 Recess
6 End 36 Orifice
7 End 37 Guide projection
8 Terminal wall 38 Guide projection
9 Closure device 39 Guide web
10 Interior 40 Guide web
11 Separating device 41 Base body
12 Container wall 42 Seal mechanism
13 Wall thickness 43 Filter element
14 Dimension 44 Flow passage
15 Longitudinal axis 45 End region
16 Plane 46 End region
17 Plane 47 Cut-out
18 Dimension 48 Insert part
19 Internal wall 49 Distance
20 Cap 50 Plane
21 Seal mechanism 50a Plane
22 Sealing stopper 51 Dimension
23 Cap case 52 Dimension
24 Coupling part 53 Cone angle
25 Coupling part 54 Gap
26 Coupling part 55 Arc length
27 Coupling part 56 Gap face
28 Coupling mechanism 57 Gap face
29 Projection 58 Width
30 Projection 59 Depth
60 Retaining mechanism 93 Positioning mechanism
61 Web 94 Stop surface
62 Length 95 Hollow cylinder segment
63 Recess 96 Terminal wall part
64 Casing part 97 Retaining web
65 Orifice 98 Connecting part
66 Wall part 99 Guide part
67 Rib 100 Retaining orifice
68 Groove 101 Retaining projection
69 Displacement path 102 Inner container
70 Dimension 103 Terminal end
71 Plane 104 Hinge joint
72 Dimension 105 Terminal end
73 Seal mechanism 106 Terminal end
74 Base body 107 Passage
75 Pressing element 108 Inlet orifice
76 Dimension 109 Center
77 Dimension 110 Peripheral region
78 Plane of symmetry 111 Base body
79 Resilient web 112 Projection
80 Cut-out 113 Seal element
81 Seal surface 114 External dimension
82 Seal arrangement 115 Energy-directing transmitter
83 Sealing lip 116 Wall part
84 External surface 117 Recess
85 Support element 118 Snap hook
86 Cone portion 119 Housing
87 Baffle surface 120 Cut-out
88 Flow surface 121 Wall part
89 Support body 122 Retaining shoulder
90 Retaining mechanism 123 Retaining lug
91 Shoulder 124 Seal lug
92 Web 125 Shoulder
126 Cut-out
127 Sealing lip
128 Guide element
129 Contact surface
130 Insert element
131 Baffle element
132 Cone angle
133 Separating element
134 Passage
135 Distance

1-139. (canceled)

140. Method of separating body fluid, such as blood, into its lighter and heavier constituents, whereby a housing unit (1) comprising a housing container (5), at least one closure device (9) for an open end (6) and a separating device (11) inserted in an interior (10) can be provided, and the separating device (11) has at least one base body (41, 74), at least one flow passage (44) extending between mutually spaced end regions (45, 46), and a filter element (43) or separating element (133) with passages (107, 134) extending between terminal ends (105, 106) which establish a flow connection with the flow passage (44), and the separating device (11) is disposed directly adjacent to the closure device (9) in its initial position, and the body fluid to be separated is then introduced into the interior (10), after which the filled housing unit (1) is centrifuged, during which the separating device (11) is moved from its initial position into an operating position spaced at a distance apart therefrom, wherein in order to introduce the body fluid into the interior (10), the filter element (43) or the separating element (133) of the separating device (11) is pierced by means of a cannula starting from the closure device (9) forming an inlet orifice (108) and forcing and piercing the elastic material, and once the cannula has been removed from the filter element (43) or separating element (133), the inlet orifice (108) produced in it by the cannula is closed for the most part due to the elastically rebounding material and the constituents are thus prevented from passing through the inlet orifice (108).

141. Method according to claim 140, wherein the passages (134) disposed in the separating element (133) are designed so that a flow of the constituents of the body fluid to be separated is permitted exclusively whilst a pressure force is being applied to the body fluid until an operating position is reached, and a flow through the passages (134) by the constituents to be separated is prevented in the operating position.

142. Method according to claim 140, wherein the individual passages (107) in the filter element (43) each have a flow cross-section which permits a passage exclusively for the first lighter part of the constituents to be separated from the body fluid, such as serum or plasma.

143. Method according to claim 140, wherein the individual passages (107) in the filter element (43) each have a flow cross-section which prevents the passage of another and heavier part of the constituents of the body fluid to be separated, such as cellular constituents of the blood.

144. Method according to claims 140, wherein, the individual passages (107) in the filter element (43) permit a passage for the first lighter part of the constituents of the body fluid to be separated only for the time that a centrifugal force which acts on the filter element (43) during the separation process is being applied.

145. Method according to claim 140, wherein the passages (107, 134) of the filter element (43) or separating element (133) prevent any passage for the constituents to be separated in the initial position.

146. Method according to claim 140, wherein the passages (107, 134) in the filter element (43) or separating element (133) prevent a passage for the constituents to be separated from the body fluid in the operation position.

147. Method according to claim 140, wherein the passages (107, 134) allow air to pass through, at least in the initial position.

148. Method according to claim 140, wherein the elastic material of the filter element (43) or separating element (133) is selected from the group comprising polyolefins, such as polyethylene or polypropylene, polyamide, polystyrene, polyether sulfane, polyester, thermoplastic elastomers (TPE, TPU), glass fibers, cellulose or compounds of these, for example mixed cellulose esters, cellulose acetate, cellulose nitrates, other natural fibers, such as for example cotton fibers, or a combination of these.

149. Method according to claim 140, wherein the filter element (43) or separating element (133) has a differing density starting from its center (109) through to its peripheral region (110) which can be applied to the housing container (5).

150. Method according to claim 140, wherein the density of the filter element (43) or separating element (133) is higher at its center (109) than the density in the peripheral region (110).

151. Method according to claim 140, wherein the density of the filter element (43) or separating element (133) is lower at its center (109) than the density in the peripheral region (110).

152. Method according to claim 140, wherein the base body (41, 74) extends out from the filter element (43) or separating element (133) towards a side facing away from the longitudinal axis (15).

153. Method according to claim 140, wherein in that at least certain regions of the filter element (43) or separating element (133) are surrounded by the base body (41, 74).

154. Method according to claim 140, wherein the filter element (43) or separating element (133) is completely surrounded by the base body (41, 74) in its region of the circumference facing the housing container (5).

155. Method according to claim 140, wherein at least certain regions of the base body (41, 74) are provided with a coating.

156. Method according to claim 140, wherein at least certain regions of the filter element (43) or separating element (133) are provided with a coating.

157. Method according to claim 140, wherein at least certain regions of the passages (107, 134) of the filter element (43) or separating element (133) are provided with a coating.

158. Method according to claim 140, wherein the coating is a chemical substance, in particular selected from the group comprising silicone, silicone oil, or based on nano-technology, in particular nano-particles.

159. Method according to claim 140, wherein the base bodies (74) can be pressed against the internal wall (19) of the housing container (5) in certain regions by means of at least one pressing element (75), such as resilient webs, helical springs for example, in the initial position and the flow passage (44) is established between the base bodies (74), disposed adjacent and mutually spaced by means of the pressing element (75).

160. Method according to claim 140, wherein in the initial position, a gap (54) is formed in the base body (41) extending between the two end regions (45, 46) and widening from its center to an external surface, and the flow passage (44) can be closed in the operating position by at least one automatically acting valve arrangement.

161. Method according to claim 160, wherein the widening gap (54) is wedge-shaped.

162. Method according to claim 140, wherein the interior (10) of the housing container (5) is surrounded by an internal wall (19) and an internal dimension (14) of the housing container (5) in the region of the first open end (6) in a plane (16) oriented perpendicular to the longitudinal axis (15) is bigger than an internal dimension (18) in the region of the other end (7) in a plane (17) parallel therewith in the same spatial direction, and the flow passage (44) which is open in the initial position is sealed by the reduction in the internal dimension (14, 18) during the displacement of the separating device (11) and hence closed on reaching the operating position.

163. Method according to claim 140, wherein the separating device (11) is retained on the housing container (5) and/or on the closure device (9) with a pre-definable force in the initial position, and the separating device (11) is moved from the initial position into the operating position by applying centrifugal force which acts on the separating device (11) during the separation process after simultaneously overcoming the retaining force when the process of separating the constituents to be separated from the substance is being run.

164. Method according to claim 140, wherein a positioning mechanism (93) is provided on the housing container (5) for the separating device (11) in the region of the operating position.

165. Method according to claim 140, wherein the interior (10) of the housing container (5) is evacuated to a pressure below atmospheric pressure.

166. Method of separating body fluids, such as blood, into its lighter and heavier constituents, whereby a housing unit (1) comprising a housing container (5), at least one closure device (9) for an open end (6) and a separating device (11) inserted in an interior (10) can be provided, and the body fluid to be separated is then introduced into the interior (10), after which the filled housing unit (1) is centrifuged, during which the separating device (11) is moved from its initial position into an operating position spaced at a distance apart therefrom, wherein before introducing the body fluid to be separated, the separating device (11) with the separating element (133) is moved by a pre-definable distance (135) starting from the open end (6) of the housing container (5) from which it is filled, and is retained in this position relative to the housing container (5) (initial position), thereby forming separate part-chambers disposed on either side of the separating device (11), after which the entire interior (10) of the housing container (5) is reduced to a pressure below the external atmospheric pressure and then the interior (10) is closed, after which the substance is introduced into the first part-chamber between the separating device (11) and the open end (6) of the housing container (5) used for filling, and due to a pressure force acting on the substance, the major part of the constituents to be separated from the substance is moved through passages (134) of the separating element (133) into the other part-chamber between the separating device (11) and the other end (7) of the housing container (5), and the housing unit (1) is then exposed to a centrifugal force acting in the direction towards the other end (7), so that the substance to be separated is split into the constituents on the one hand and the separating device (11) is moved from the initial position into an operating position on the other hand, and when the centrifugal force is removed and the separating device (11) is in the operating position, the flow through the passages (134) in the separating element (133) for the constituents to be separated is blocked.

167. Method according to as claim 161, wherein the flow of the constituents of the substance through the passages (134) of the separating element (133) is blocked in both flow directions in the operating position.

168. Method according to claim 166, wherein the separating device (11) is held stationary in a pre-definable position relative to the housing container (5) when the operating position is reached.

169. Method according to claim 166, wherein at least part-surfaces of the internal wall (19) of the housing container (5) are provided with a coating.
